(19) United States
(12) Patent Application Publication
(10) Pub. No.: US 2025/0195608 A1
(43) Pub. Date: Jun. 19, 2025

(54) Therapeutic Compositions for the Treatment of Dry Eye Disease

(71) Applicant: Schepens Eye Research Institute, Boston, MA (US)

(72) Inventors: Reza Dana, Cambridge, MA (US); Sunil Chauhan, Somerville, MA (US)

(21) Appl. No.: 18/823,679

(22) Filed: Sep. 4, 2024

Related U.S. Application Data
(63) Continuation of application No. 17/152,111, filed on Jan. 19, 2021, now abandoned.
Application No. 17/152,111 is a division of application No. 15/987,694, filed on May 23, 2018, now Pat. No. 10,940,179.
Application No. 15/987,694 is a continuation of application No. 14/642,107, filed on Mar. 9, 2015, now abandoned.
Application No. 14/642,107 is a division of application No. 13/035,695, filed on Feb. 25, 2011, now Pat. No. 9,011,861.
(60) Provisional application No. 61/308,091, filed on Feb. 25, 2010.
(60) Provisional application No. 61/329,845, filed on Apr. 30, 2010.
(60) Provisional application No. 61/331,278, filed on May 4, 2010.

(51) Int. Cl. A61K 38/13; A61K 9/00; A61K 31/404; A61K 31/44; A61K 31/4439; A61K 31/444; A61K 31/502; A61K 31/506; A61K 31/517; A61K 38/12; A61K 38/17; A61K 39/00; A61K 39/395; A61K 45/06; C07K 16/22

(52) U.S. Cl. CPC A61K 38/13; A61K 9/0048; A61K 31/404; A61K 31/44; A61K 31/4439; A61K 31/444; A61K 31/502; A61K 31/506; A61K 31/517; A61K 38/12; A61K 38/179; A61K 39/3955; A61K 45/06; C07K 16/22; A61K 2039/505; C07K 2317/76

(57) ABSTRACT

Described herein are materials and methods for treating dry eye disease in a subject.

### CROSS-REFERENCE TO RELATED APPLICATIONS

This application is a continuation application of U.S. patent application Ser. No. 17/152,111, filed on Jan. 19, 2021, abandoned; which is a divisional of U.S. patent application Ser. No. 15/987,694, filed on May 23, 2018, now U.S. Pat. No. 10,940,179; which is a continuation of U.S. patent application Ser. No. 14/642,107, filed Mar. 9, 2015, abandoned; which is a divisional of U.S. patent application Ser. No. 13/035,695, filed on Feb. 25, 2011, now U.S. Pat. No. 9,011,861; which claims the benefit of priority under § 119 of U.S. Provisional Application Nos. 61/308,091, 61/329,845, and 61/331,278, filed on Feb. 25, 2010, Apr. 30, 2010, and May 4, 2010, respectively. The disclosure of each of the priority applications is incorporated herein by reference in its entirety.

### FEDERALLY SPONSORED RESEARCH OR DEVELOPMENT

This invention was made with Government support under Grant Number EY012963, awarded by the National Institute of Health. The Government has certain rights in the invention.

### FIELD OF THE INVENTION

This invention relates generally to the field of ophthalmology.

### INCORPORATION BY REFERENCE OF MATERIALS SUBMITTED ELECTRONICALLY

This application contains, as a separate part of the disclosure, a Sequence Listing in computer readable form (Filename: 45367A4_SubSeqlisting.xml; Size: 83,312 bytes; Created: Oct. 28, 2024), which is incorporated by reference in its entirety.

### BACKGROUND OF THE INVENTION

Dry Eye Disease (DED) is a relatively common condition characterized by inadequate tear film protection of the cornea. Dry eye symptoms have traditionally been managed with eyelid hygiene, topical antibiotics (erythromycin or bacitracin ointments), oral tetracyclines (tetracycline, doxycycline, or minocycline), anti-inflammatory compounds (cyclosporine) and corticosteroids which are often time consuming, frustrating, and frequently ineffective or variably effective treatments.

Tens of millions of people (mostly women) are affected worldwide by dry eye. 10 million people in US are affected with severe dry eyes with more than 3.2 million women and 1.6 million men above the age of 50 years being affected by dry eye in the US. DED is a potentially disabling disease adversely impacting the vision-related quality of life. It leads to ocular discomfort, a degradation in visual performance (reading speed, contrast sensitivity) and a loss of productivity. Current therapeutic options are limited and costly. Topical cyclosporine-A (Restasis®) is the only approved treatment for DED in US. Despite the high incidence of DED, there is currently no consistently effective treatment for this condition and it still remains a therapeutic challenge. As such, there is a need for new therapeutic modalities to treat DED.

### SUMMARY OF THE INVENTION

The present invention discloses a novel method for the treatment of dry eye disease in humans comprising local application of an anti-lymphangiogenic agent onto the ocular surface. The present invention is based on novel evidence for the selective growth of lymphatic vessels in DED cornea. Additionally, significant increase in both caliber and extent of lymphatics in DED corneas is accompanied by over expression of lymphangiogenic receptor VEGFR-3, further correlating DED with lymphangiogenesis.

An anti-lymphangiogenic agent of the invention is selected from the group consisting of: a nucleic acid molecule, an aptamer, an antisense molecule, an RNAi molecule, a protein, a peptide, a cyclic peptide, an antibody or antibody fragment, a polysaccharide, and a small molecule.

In one preferred embodiment of the invention, the anti-lymphangiogenic agent is an inhibitor of VEGF-C or VEGF-D mediated signal transduction by VEGFR-2 or VEGFR-3. Preferably, the amount of the anti-lymphangiogenic agent employed is effective to inhibit the binding of VEGF-C and/or VEGF-D ligand to VEGFR-3 or the stimulatory effect of VEGF-C and/or VEGF-D on VEGFR-3.

In one aspect of the invention, the inhibitor of VEGF-C or VEGF-D mediated signal transduction by VEGFR-2 or VEGFR-3 is a molecule such as but not restricted to an antibody, a small molecule or a peptide that prevents binding of VEGF-C or VEGF-D to the receptors VEGFR-2 or VEGFR-3.

In another aspect of the invention, the inhibitor of VEGF-C or VEGF-D mediated signal transduction is a VEGFR-2 or VEGFR-3 soluble receptor. Soluble receptors of VEGFR-2 or VEGFR-3 can be administered directly. Alternatively, increase in the secretion of VEGFR-2 or VEGFR-3 is accomplished by inserting the VEGFR-2 or VEGFR-3 soluble receptors genes into the genome of corneal cells. This could be epithelial cells, keratocytes, fibroblasts, endothelial cells, or bone marrow-derived cells. Methods to introduce genes into a genome of a cell are well-known in the art. Genes are introduced in the genome of corneal cells using viral or non-viral vectors. Viral vectors include for example adenoviruses, retroviruses or lentiviruses. Non-viral vectors include, for example, liposomes such as cationic lipids, nanoparticles, lipoplexes and polyplexes (complexes of polymers with DNA).

In some embodiments, the anti-lymphangiogenic agent is a VEGF-C antibody, wherein the antibody comprises a heavy chain variable region set forth in amino acids 1-118 of SEQ ID NO: 34 or a heavy chain comprising the amino acid sequence of SEQ ID NO: 34. In some embodiments, the VEGF-C antibody is selected from the group consisting of antibodies 69D09, 103, MM0006-2E65 and 193208.

In alternative embodiments, the anti-lymphangiogenic agent is antibody that competitively inhibits the binding of antibody 69D09 to VEGF-C.

In some embodiments, the anti-lymphangiogenic agent is a VEGF-D antibody selected from the group consisting of antibodies 2F8, 4A5 (VD1), 4E10, 5F12, 4H4, 3C10 28AT743.288.48, MM0001-7E79, RM0007-8C35, 78902, 78939 and 90409.

In some embodiments, the anti-lymphangiogenic agent is a human or humanized antibody.

In other embodiments, the anti-lymphangiogenic agent is a soluble VEGFR-3 fragment that binds VEGF-C or VEGF-D.

In still other embodiments, the anti-lymphangiogenic agent is a VEGFR-2 inhibitor.

In one embodiment of the invention, the anti-lymphangiogenic agent is administered in combination with an anti-inflammatory agent such as, but not limited to, a composition inhibiting the activity of an inflammatory cytokine selected from the group comprising IL-1,IL-17, TNF-α and IL-6.

Exemplary functional blockers of IL-1 are described in WO/2009/025763. Exemplary functional blockers of TNF-α include, but are not limited to, recombinant and/or soluble TNF-α receptors, monoclonal antibodies, and small molecule antagonists and/or inverse agonists. One or more commercially-available TNF-α blocking agents are reformulated for topical administration in this embodiment. Exemplary commercial TNF-α blocking agents used for reformulation include, but are not limited to, etanerept/Ernbrel, infliximab/Remicade, and adalimumab/Humira.

In one embodiment of the invention, the anti-lymphangiogenic agent is administered in combination with an antiobiotic. Exemplary antibiotic compositions used for combination-therapy with antagonists of IL-mediated inflammation include but are not limited to, amikacin, gentamicin, kanamycin, neomycin, netilmicin, streptomycin, tobramycin, teicoplanin, vancomycin, azithromycin, clarithromycin, dirithromycin, erythromycin, roxithromycin, troleandomycin, amoxicillin, ampicillin, azlocillin, carbenicillin, cloxacillin, dicloxacillin, flucloxacillin, meziocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, oflaxacin, trovafloxacin, mafenide, sulfacetamide, sulfamethizole, sulfasalazine, sulfisoxazole, trimethoprim, cotrimoxazole, demeclocycline, doxycycline, minocycline, oxytetracycline, or tetracycline.

The composition of the invention is locally applied to the ocular tissue, alternatively the composition of the invention is applied to the eyelids, the ocular surface, the meibomian glands or the lacrimal glands.

The composition can be in the form of a solid, a paste, an ointment, a gel, a liquid, an aerosol, a mist, a polymer, a film, an emulsion, or a suspension.

Optionally, the composition further contains a compound selected from the group consisting of a physiological acceptable salt, poloxamer analogs with carbopol, carbopol/hydroxypropyl methyl cellulose (HPMC), carbopol-methyl cellulose, carboxymethylcellulose (CMC), hyaluronic acid, cyclodextrin, and petroleum.

Dry eye disease may be attributable to a number of factors, and treatment of subjects who have developed dry eye disease due to a variety of specific factors is contemplated. In some variations, the DED to be treated is DED caused by any condition other than an alloimmune response. Alloimmune responses may result, for example, in some corneal transplant patients. More specifically, in some variations, the DED to be treated is an autoimmune DED or a DED associated with Sjogren's syndrome. In some variations, the DED is due to excessively fast tear evaporation (evaporative dry eyes) or inadequate tear production. In some variations, the dry eye disease is attributable to one or more causes selected from: aging, contact lens usage and medication usage. In some variations, the dry eye disease is a complication of LASIK refractive surgery. In other variations, the DED arises in a subject who has not had eye surgery of any kind, e.g., treatment of subjects in whom the DED is caused by LASIK surgery, corneal transplant surgery, or other ocular surgeries.

### DESCRIPTION OF THE DRAWINGS

FIG. 1: Representative whole mount corneal immunofluorescence micrographs showing lymphatics (CD31^{lo}/LYVE-1^{hi}) in normal and dry eye (DE) at day 14 (20× magnification).

FIG. 2: Representative whole mount corneal immunofluorescence micrographs showing lymphatics (CD31^{lo}/LYVE-1^{hi}) in normal and dry eye (DE) at days 6, 10 and 14 (100× magnification).

FIGS. 3A-3B: Quantification of lymphatics in dry eye (DE) corneas. Morphometric analysis of corneal lymphangiogenesis in normal and DE days 6, 10 and 14 (100× magnification). Morphometric evaluation showed significant increase in lymphatic area (LA) in dry eye compared to normal corneas (FIG. 3A). Significant increase in lymphatic caliber (LC) in dry eye compared to normal corneas was noticed only at day 14 (FIG. 3B). Data from a representative experiment of three performed is shown as mean±S.E.M and each group consists of four to five mice.

FIG. 4: Analysis of lymphangiogenic-specific growth factors. Real-time PCR analysis showing transcript levels of VEGF-A, VEGF-C and VEGF-D in the dry eye corneas at different time points. A significant increase in VEGF-D was seen at day 6 whereas VEGF-A and VEGF-C increased significantly only by day 14. Data from a representative experiment of three performed is shown as mean±S.E.M and each group consists of four to five mice.

FIG. 5: Analysis of lymphangiogenic-specific growth factor receptors. Real-time PCR analysis showing transcript levels of VEGFR-2 and VEGFR-3 in the dry eye corneas at different time points. Significant increase in VEGFR-3 was seen earliest at day 6 but VEGFR-2 increased significantly later in disease at day 14. Data from a representative experiment of three performed is shown as mean±S.E.M and each group consists of four to five mice.

FIG. 6: Enumeration of corneal CD11b⁺/LYVE-1⁺ cells. A significant increase in the number of both CD11b⁺ and double stained CD11^{hi}/LYVE-1⁺ cells in the dry eye corneas as compared to normal. Data from a representative experiment of three performed is shown as mean±S.E.M and each group consists of four to five mice.

FIG. 7: Increased homing of mature MTIC-II+CDIlb+ APC in the draining LN of DED mice. Flow cytometric analysis of draining lymph nodes showing significant increase in the frequencies of mature MHC-II+CD11b⁺ APC in DED mice compared with normal mice. Data from a representative experiment of two performed is shown and each group consists of three mice.

FIG. 8: Analysis of lymphangiogenic-specific growth factors and their receptors. Real-time PCR analysis showing transcript levels of VEGF-A, VEGF-C, VEGF-D, VEGFR-2 and VEGFR-3 in the dry eye corneas.

FIG. 9: Analysis of proinflammatory cytokines in conjunctiva. Real-time PCR analysis showing expression of cytokines IL-1α, IL-1β, IL-6, IL-17. The levels of all four cytokines in the conjunctiva showed significantly decreased expression in anti-VEGF-C treated DED mice as compared to those of untreated DED mice

FIG. 10: Analysis of inflammatory cytokines in draining lymph nodes. Real-time PCR analysis for IL-17 (Th 17 cells) and IFN-γ (Th1 cells). Draining lymph nodes of anti-VEGF-C treated DED mice showed significantly decreased induction of T-cell mediated autoimmune response compared untreated DED mice.

FIG. 11: Enumeration of CD11b⁺ cells in DED corneas. Treatment with anti-VEGF-C antibodies significantly decreased infiltration of CD11b⁺ cells (30%) in the DED corneas (day 14).

FIG. 12: Epifluorescent microscopic image of corneal wholemounts immunostained with CD31 and LYVE-1.

FIG. 13: Quantification of number of infiltrating CD11b⁺ cells per mm2 of cornea.

FIG. 14: In vivo blockade of VEGF-C ameliorates clinical signs of DED. Corneal fluorescein staining (CFS) score is used as readout for the clinical signs of dry eye inflammation, in anti-VEGF-C Ab-treated and untreated mice. CFS scores were significantly decreased in the group treated with anti-VEGF-C antibody at days 5, 9 and 13 vs the untreated group. Data shown as mean±S.E.M and each group consisted of 3-4 mice.

### DETAILED DESCRIPTION

### Dry Eye

Keratoconjunctivitis sicca (KCS), also called keratitis sicca, sicca syndrome, xerophthalmia, dry eye syndrome (DES), or simply dry eyes, is an eye disease caused by decreased tear production or increased tear film evaporation commonly found in humans and some animals. Typical symptoms of keratoconjunctivitis are dryness, burning and a sandy-gritty eye irritation that gets worse as the day goes on.

Keratoconjunctivitis sicca is characterized by inadequate tear film protection of the cornea because of either inadequate tear production or abnormal tear film constitution, which results in excessively fast evaporation or premature destruction of the tear film. The tear film is constituted by 3 layers: (1) a lipid layer, produced by the Meibomian glands; (2) an aqueous layer, produced by the main and accessory lacrimal glands; and (3) a hydrophilic mucin layer, produced by the conjunctival goblet cells. Any abnormality of 1 of the 3 layers produces an unstable tear film and symptoms of keratitis sicca.

Sjogren's syndrome and autoimmune diseases associated with Sjogren's syndrome are also conditions associated with aqueous tear deficiency. Drugs such as isotretinoin, sedatives, diuretics, tricyclic antidepressants, antihypertensives, oral contraceptives, antihistamines, nasal decongestants, beta-blockers, phenothiazines, atropine, and pain-relieving opiates such as morphine can cause or worsen this condition. Infiltration of the lacrimal glands by sarcoidosis or tumors, or post-radiation fibrosis of the lacrimal glands can also cause this condition.

Keratoconjunctivitis sicca can also be caused by abnormal tear composition resulting in rapid evaporation or premature destruction of the tears. When caused by rapid evaporation, it is termed evaporative dry eyes. In this, although the tear gland produces a sufficient amount of tears, the rate of evaporation of the tears is too rapid. There is a loss of water from the tears that results in tears that are too “salty” or hypertonic. As a result, the entire conjunctiva and cornea cannot be kept covered with a complete layer of tears during certain activities or in certain environments.

Aging is one of the most common causes of dry eyes. About half of all people who wear contact lenses complain of dry eyes. There are two potential connections between contact lens usage and dry eye. Traditionally, it has been believed that soft contact lenses, which float on the tear film that covers the cornea, absorb the tears in the eyes. However, it is also now known that contact lens usage damages corneal nerve sensitivity, which may lead to decreased lacrimal gland tear production and dry eye. The effect of contact lenses on corneal nerve sensitivity is well established for hard contact lenses as well as soft and rigid gas permeable contact lenses. The connection between this loss in nerve sensitivity and tear production is the subject of current research. Dry eyes also occur or get worse after LASIK and other refractive surgeries. The corneal nerves stimulate tear secretion. Dry eyes caused by these procedures usually resolves after several months. Persons who are thinking about refractive surgery should consider this.

A variety of approaches can be taken to treat dry eyes. These can be summarized as: avoidance of exacerbating factors, tear stimulation and supplementation, increasing tear retention, eyelid cleansing and treatment of eye inflammation. Application of artificial tears every few hours can provide temporary relief. Inflammation occurring in response to tears film hypertonicity can be suppressed by mild topical steroids or with topical immunosuppressants such as cyclosporine. Consumption of dark-fleshed fish containing dietary omega-3 fatty acids is associated with a decreased incidence of dry eyes syndrome in women. Early experimental work on omega-3 has shown promising results when used in a topical application (Rashid S et al (2008). Arch Ophthalmol126 (2): 219-225).

DED is increasingly recognized as an immune-mediated disorder. Desiccating stress in DED initiates an immune-based inflammation response that is sustained by the ongoing interplay between the ocular surface and various pathogenic immune cells, primarily the CD4⁺ cells in the conjunctiva and the CD11b⁺ monocytic cells in the corneal. Desiccating stress induces secretion of inflammatory cytokines, especially IL-1, TNF-α and IL-6 by ocular tissues, which facilitates the activation and migration of resident antigen presenting cells (APCs) toward the regional draining lymph nodes (LNs). In the LNs, these APCs stimulate naive T-cells, leading to the expansion of IL-17 secreting Th17 cells and interferon (IFN)-γ-secreting Th1 cells. Once these effectors are generated in the LNs, they migrate to the ocular surface and secrete effector cytokines.

### VEGF

VEGF (Vascular Endothelial Growth Factor) is a sub-family of growth factors, specifically the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature). Members of the platelet-derived growth factor family include the Placenta growth factor (PIGF), VEGF-A (also known as VEGF), VEGF-B, VEGF-C, VEGF-D and VEGF-E.

VEGF-A, VEGF-C and VEGF-D exert their effects by variously binding to and activating structurally related membrane receptor tyrosine kinases; VEGF receptor-1 (VEGFR-1 or Flt-I), VEGFR-2 (flk-1 or KDR), and VEGFR-3 (Flt-4). Members of the VEGF family may also interact with the structurally distinct receptor neuropilin-l. Binding of a VEGF to these receptors initiates a signaling cascade, resulting in effects on gene expression and cell survival, proliferation, and migration.

VEGF-A binds to VEGFR-1 (Flt-1) and to VEGFR-2 (KDR/Flk-1). VEGFR-2 appears to mediate almost all of the known cellular responses to VEGF-A. The function of VEGFR-1 is less well-defined, although it is thought to modulate VEGFR-2 signaling. VEGF-A is believed to play a central role in the development of new blood vessels (angiogenesis) and the survival of immature blood vessels (vascular maintenance).

VEGF-C and VEGF-D are ligands for VEGFR-2 and VEGFR-3 and are involved in the mediation of lymphangiogenesis.

### Lymphangiogenesis

Lymphangiogenesis refers to formation of lymphatic vessels, particularly from pre-existing lymphatic vessels, but as used herein, the term applies to formation of lymph vessels under any condition. It also applies to the enlargement of lymphatic vessels, commonly known as lymphatic hyperplasia. Lymphangiogenesis plays an important physiological role in homeostasis, metabolism and immunity. Lymphatic vessel formation has also been implicated in a number of pathological conditions including neoplasm metastasis, oedema, rheumatoid arthritis, psoriasis and impaired wound healing.

The normal human cornea is avascular, thus suppressing the afferent lymphatic and efferent vascular arms or the immune cycle. Inflammation however negates this immune and angiogenic privileged state of the cornea and giving the corneal and ocular surface the potential to mount an immune response. Our results show that corneal lymphatics play an important role in mediating the corneal inflammation in dry eyes. Inhibition of corneal lymphangiogenesis decreases ocular surface inflammation in a well characterized mouse model of DED.

Lymphangiogenesis is regulated to a large extent by VEGF-C and VEGF-D. Lymphangiogenesis appears to be regulated by signaling mediated by VEGFR-3, particularly upon specifically binding its ligands, VEGF-C and VEGF-D. VEGF-C and VEGF-D are two of six members of a family of angiogenic regulators. Other members are VEGF-A (also known as VEGF), VEGF-B, VEGF-E and placental growth factor (PIGF).

During embryogenesis, lymphatic endothelial cell sprouting, proliferation and survival is promoted by VEGF-C. Lymphatic vessels fail to develop in mice in which VEGF-C is absent (Vegfc knockout mice), and such mice develop severe edema. Indeed, absence of VEGF-C is embryonic lethal. Lymphatic vessel hypoplasia and lymphedema is exhibited in the skin of mice hemizygous for Vegfc (i.e. mice possessing one functional allele).

Lymphangiogenesis is also partly regulated by VEGF-D, similar to VEGF-C. However, lymphangiogenesis during embryonic development is not dependent upon VEGF-D, as demonstrated by Vegfd knockout mice. The lymphatic system in Vegfd knockout mice is relatively normal and Vegfd knockout mice are viable and fertile. The absolute abundance of lymphatic vessels in the lung is, however, reduced by approximately 30% compared to wild-type mice.

Lymphatic vessels express VEGFR-3, the receptor for VEGF-C and VEGF-D, and both VEGF-C and VEGF-D signal predominantly through VEGFR-3. It is also becoming apparent that lymphatic vessels variously express VEGFR-2. VEGF-C and VEGF-D are synthesized as prepro-polypeptides and are proteolytically processed by proprotein convertases. In humans, mature proteolytically processed forms of VEGF-C and VEGF-D bind to VEGFR-2 and VEGFR-3. In mice, mature VEGF-D binding is restricted to VEGFR-3.

VEGF-C and VEGF-D exist as homodimers, and it has been suggested that they may exist as VEGF-C-VEGF-D heterodimers. In addition to lymphatic vessels, VEGFR-3 is also expressed on blood vessel endothelial cells during development, thereby accounting for the severe vasculogenic and angiogenic defects observed during early embryogenesis in models comprising inactive VEGFR-3 signaling. The lymphatic system possesses almost exclusive expression of VEGFR-3 in healthy tissues in adulthood, because VEGFR-3 expression in blood vessels declines following birth and during adolescence. Thus, only lymphangiogenesis is inhibited in adults by inhibition of the VEGF-C-VEGF-D-VEGFR-3 signaling axis.

Lymphatic vessels express neuropilin-2 (NRP-2), which can bind VEGF-C or VEGF-D. In lymphangiogenesis, NRP-2 is thought to act as a co-receptor to increase the binding affinity of VEGF-C or VEGF-D to VEGFR-3. NRP-2 is required for lymphangiogenesis. Proliferation of lymphatic vessel endothelial cells was reduced and lymphatic vessels and capillaries failed to develop in Nrp2 knockout mice in which NRP-2 is absent. Similarly, NRP-1 is capable of binding VEGF-C and VEGF-D.

Defective lymphatic capillaries are the underlying cause of Milroy disease and other rare hereditary forms of lymphedema in humans. Tyrosine kinase-inactivating point mutations of the VEGFR-3 gene have been identified as a major cause of Milroy disease, and VEGF-C and VEGF-D therapy has shown promising efficacy in preclinical animal models. However, previous work has only demonstrated lymphatic capillary reconstitution, whereas effects on the collecting lymphatic vessels that are more commonly damaged in lymphedema have not been addressed.

Lymphatic vessel growth in adult tissues can be induced by Angiopoietin-1 (ANG-1) through its binding to the tunica interna endothelial cell kinase receptor 2 (TIE-2 or TEK). Lymphatic vessel sprouting that was induced by ANG-1 was inhibited by an inhibitor of VEGFR-3. Furthermore, VEGFR-3 was up-regulated by ANG-1 binding to TIE-2. TIE-2 expressed on lymphatic vascular endothelial cells may also be agonized by ANG-2 and ANG-3.

VEGF-C and VEGF-D may act as ligands for integrins. Specifically, VEGF-C and VEGF-D have been shown to act as ligands for integrin α9β1. Cell adherence and cell migration were promoted by each of VEGF-C and VEGF-D in cells expressing integrin 19 [1. The effect could be blocked by an anti-integrin α9β1 antibody or siRNA directed to integrin α9ß1.

Thus, in lymphangiogenesis, VEGFR-3 appears to be central. VEGFR-3 specifically binds and is activated by ligands VEGF-C and VEGF-D. VEGF-C and VEGF-D are synthesized as prepro-polypeptides and are activated by proteolytic processing by proprotein convertases. VEGF-C and VEGF-D also bind specifically to NRP-2, which is thought to be a co-receptor for VEGFR-3. Both lymphangiogenesis and VEGFR-3 are up-regulated when ANG-1 specifically binds to TIE-2. It is thought that binding of VEGF-C or VEGF-D to integrins, particularly integrin α9ß1, also performs a role in lymphangiogenesis.

Lymphangiogenesis is mediated primarily by the interaction of growth factors VEGF-C and VEGF-D on VEGFR-2 and VEGFR-3, and in particular VEGFR-3. VEGF-A also contributes, albeit indirectly, to lymphangiogenesis by recruiting VEGF-C and VEGF-D secreting macrophages. Inhibition of VEGF-C and VEGF-D signaling pathways would thus constitute a new approach to the treatment of DED. The invention is however not restricted to the inhibition of VEGF-C and VEGF-D signaling pathways and according to the present invention, other anti-lymphangiogenic agents can be used to reduce the signs and symptoms of DED.

### Anti-Lymphangiogenic Agents

Persons skilled in the art will appreciate from the foregoing that inhibition of lymphangiogenesis can occur at a variety of biological points comprising any one or more of the interactions described. For example, inhibition may occur by targeting VEGF-D, VEGF-C or VEGFR-3.

An “anti-lymphangiogenic agent” is any substance that partially or fully blocks, neutralizes, reduces, inhibits or antagonizes a biological activity of a molecular component of signaling mediated by VEGFR-3 or lymphangiogenesis. Alternatively, an anti-lymphangiogenic agent is any substance that partially or fully blocks, neutralizes, reduces, inhibits or antagonizes a VEGF-C or VEGF-D biological activity. Thus, “inhibition” is the corresponding state elicited by an inhibitor. A molecular component of signaling mediated by VEGFR-3 or lymphangiogenesis includes VEGFR-3, VEGFR-2, VEGF-C, VEGF-D, proprotein convertases, neuropilin-1 (NRP-1), neuropilin-2 (NRP-2), angiopoietin-1 (ANG-1), tunica interna endothelial cell kinase receptor (TIE-2) or integrin α9ß1.

It is envisaged that practice of the invention extends to any inhibitor known now or in the future.

Suitable classes of inhibitor molecules that target VEGF-C or VEGF-D or signaling mediated by VEGFR-3, or lymphangiogenesis include antibodies, polypeptides, peptides, peptide mimetics, nucleic acid molecules, and small molecules. Such classes of inhibitor molecules are suitable also for inhibiting binding of ligands, for example VEGF-C or VEGF-D, to integrins, particularly integrin α9ß1.

Suitable VEGF-C, VEGF-D, VEGFR-3-mediated signaling or lymphangiogenesis antibody inhibitors include antagonist and neutralizing antibodies or antibody fragments.

Polypeptide, peptide, or peptide mimetic VEGF-C or VEGF-D inhibitors, VEGFR-3-mediated signaling inhibitors or lymphangiogenesis inhibitors include fragments or amino acid sequence variants of native polypeptide or peptide components of VEGF-C, VEGF-D, VEGFR-3-mediated signaling or lymphangiogenesis.

Nucleic acid molecule inhibitors of VEGF-C or VEGF-D activity, VEGFR-3-mediated signaling or lymphangiogenesis include antisense molecules, nucleic acids in triple-helix formation, small interfering RNA (siRNA), and ribozymes.

Small molecule inhibitors of VEGF-C or VEGF-D, VEGFR-3-mediated signaling or lymphangiogenesis include organic and inorganic molecules.

Inhibitors of VEGF-C or VEGF-D activity, VEGFR-3-mediated signaling or lymphangiogenesis according to the present invention may exert their effects by interacting with any one or more of VEGFR-3, VEGFR-2, VEGF-C, VEGF-D, proprotein convertases, NRP-1, NRP-2, ANG-1, TIE-2 or integrins, particularly integrin α9β1, in their DNA, RNA or polypeptide forms.

Inhibition of VEGF-C or VEGF-D activity, VEGFR-3-mediated signaling or lymphangiogenesis according to the present invention may occur via inhibition of ligand availability for receptor binding, inhibition of receptor availability for ligand binding, inhibition of receptor tyrosine kinase activity, or inhibition of co-receptor interaction.

As used herein, “availability” refers to the potential or actual amount of a molecule that performs some function in VEGF-C or VEGF-D activity, VEGFR-3-mediated signaling or lymphangiogenesis and is present in a biological system. Availability may be relative or absolute. For example, if all copies of a gene encoding a polypeptide involved in lymphangiogenesis were rendered non-functional by genetic mutation and no functioning polypeptide was synthesized, then there would be no availability of the polypeptide in an absolute sense. Alternatively, if the same gene was present with one functioning copy and 50% of the polypeptide was synthesized, there would be reduced or inhibited availability in a relative sense. Similarly, other mechanisms may be envisaged where availability is affected. Receptors may be transcribed or translated to a lesser degree when compared with a control, or the receptor may be targeted by an antibody that binds specifically to the ligand binding site, thereby reducing or inhibiting receptor availability for ligand binding. Analogously, if ligand synthesis is targeted by an antisense inhibitor, or if an antibody inhibitor or soluble receptor inhibitor specifically binds to the ligand, then there will be reduction or inhibition of ligand availability for receptor binding.

The term “specific binding” or “specifically binds” or “specific for” refers to binding where a molecule binds to a particular polypeptide or epitope on a particular polypeptide without substantially binding to any other polypeptide or polypeptide epitope. Such binding is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. As used herein, specific binding is used in relation to the interaction between the molecular components of VEGF-C or VEGF-D activity, VEGFR-3-mediated signaling or lymphangiogenesis. Specific binding is also used in relation to the interaction between the molecular components of VEGF-C or VEGF-D activity, VEGFR-3-mediated signaling or lymphangiogenesis and agents that partially or fully block, neutralize, reduce or antagonize a biological activity of a molecule that facilitates VEGFR-3-mediated signaling or lymphangiogenesis. Specific binding also applies to the interaction between the molecular components of VEGF-C or VEGF-D activity and agents that partially or fully block, neutralize, reduce or antagonize VEGF-C or VEGF-D biological activity.

In particular, specific binding refers to a molecule having a K_{d} at least 2-fold less for the particular polypeptide or epitope on a particular polypeptide than it does for a non-specific target. Preferably, specific binding refers to a molecule having a K_{d} at least 4-fold, 6-fold, 8-fold or 10-foldless for the particular polypeptide or epitope on a particular polypeptide than it does for a non-specific target. Alternatively, specific binding can be expressed as a molecule having a K_{d} for the target of at least about 104 M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or less.

The person skilled in the art will appreciate that there exist many mechanisms for inhibiting VEGF-C or VEGF-D activity, VEGFR-3-mediated signaling or lymphangiogenesis. The principal aim is to reduce receptor signaling. Some examples will be described below, but such a list is not intended to be limiting.

### Antibody Inhibitors

The term “antibody” is used in the broadest sense and specifically covers, for example, polyclonal antibodies, monoclonal antibodies (including antagonist and neutralizing antibodies), antibody compositions with polyepitopic specificity, single chain antibodies, and fragments of antibodies, provided that they exhibit the desired biological or immunological activity.

An “antibody inhibitor” will specifically bind to a particular polypeptide or epitope on a particular polypeptide without substantially binding to any other polypeptide or polypeptide epitope. Such binding will partially or fully block, neutralize, reduce or antagonize VEGF-C or VEGF-D activity or a biological activity of a molecule that facilitates VEGFR-3-mediated signaling or lymphangiogenesis. Such target molecules include VEGFR-3, VEGFR-2, VEGF-C and VEGF-D, for example.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaccous or non-proteinaccous solutes. Generally, the antibody will be purified (1) to greater than 95% by weight of antibody as determined by the Lowry method, and most preferably more than 99% by weight, (2) to a degree sufficient to obtain at least 15 residues of N terminal or internal amino acid sequence by use of a spinning cup sequenator, or (3) to homogencity by SDS PAGE under reducing or non-reducing conditions using Coomassie blue or, preferably, silver stain. An isolated antibody includes the antibody in situ within recombinant cells since at least one component of the antibody's natural environment will not be present. Ordinarily, however, isolated antibody will be prepared by at least one purification step.

Where antibody fragments are used, the smallest inhibitory fragment that specifically binds to the binding domain of the target protein is preferred.

### Polyclonal Antibodies

Polyclonal antibodies are preferably raised in animals by multiple subcutaneous (s.c.) or intraperitoneal (i.p.) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

In one protocol for generating polyclonal antibodies, animals are immunized against the antigen, immunogenic conjugate, or derivative, by combining the antigen, conjugate or derivative with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

### Monoclonal Antibodies

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method.

Monoclonal antibodies may be made using the hybridoma method in which a mouse or other appropriate host animal, such as a hamster, is immunized to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell.

The hybridoma cells thus prepared are seeded and grown in a suitable culture medium, which preferably contains one or more substances that inhibit the growth or survival of the unfused, parental myeloma cells (also referred to as fusion partner).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

Once hybridoma cells that produce antibodies of the desired specificity, affinity, and/or activity are identified, the clones may be subcloned by limiting dilution procedures and grown by standard methods. In addition, the hybridoma cells may be grown in vivo as ascites tumors in an animal e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxyapatite chromatography, gel electrophoresis, or dialysis.

DNA encoding the monoclonal antibodies is readily isolated and sequenced using conventional procedures. The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells.

Monoclonal antibodies or antibody fragments can be isolated from antibody phage libraries. High affinity (nM range) human antibodies can be generated by chain shuffling, as well as combinatorial infection and in vivo recombination as a strategy for constructing very large phage libraries. Thus, these techniques are viable alternatives to traditional monoclonal antibody hybridoma techniques for isolation of monoclonal antibodies.

The DNA that encodes the antibody may be modified to produce chimeric or fusion antibody polypeptides. The monoclonal antibodies used herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

### Human and Humanized Antibodies

The anti-VEGF-C, anti-VEGF-D, anti-VEGFR-3-mediated signaling or anti-lymphangiogenesis antibodies used in the invention may comprise humanized antibodies or human antibodies. Generally, a “humanized antibody” is an antibody of non-human origin that has been modified using recombinant DNA techniques to circumvent the problem of a human's immune system reacting to an antibody as a foreign antigen. The standard procedure of producing monoclonal antibodies produces mouse antibodies. Although murine antibodies are very similar to human ones, there are differences. Consequently, the human immune system recognizes mouse antibodies as foreign, rapidly removing them from circulation and causing systemic inflammatory effects. “Humanized” forms of non-human (e.g., rodent) antibodies are chimeric antibodies that contain a reduced percentage of sequence derived from the non-human antibody. Various forms of humanized anti-VEGF-C, anti-VEGF-D, anti-VEGFR-3-mediated signaling or anti-lymphangiogenesis antibodies are contemplated. Humanized antibodies may be intact antibodies, such as intact IgG₁ antibodies, antibody chains or fragments thereof (such as Fv, Fab, Fab′, F(ab′)2, or other antigen-binding subsequences of antibodies). Humanized antibodies include human antibodies (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human antibody are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human antibody and all or substantially all of the FR regions are those of a human antibody consensus sequence. The humanized antibody optimally also will comprise at least a portion of an antibody constant region (Fc), typically that of a human antibody.

Various humanization strategies have been described in the prior art and it is envisaged that practice of the invention extends to the use of both known humanization strategies and any new strategies to be developed in the future. Examples of known humanization strategies include those described by Studnicka (U.S. Pat. No. 5,869,619) and Padlan (1991, Molec. Immunol., 28, 489-498), Winter (U.S. Pat. No. 5,225,539) and Jones et al (1986, Nature, 321, 522-525), Queen et al. (U.S. Pat. No. 5,693,761) and Foote (U.S. Pat. No. 6,881,557).

As an alternative to humanization, human antibodies can be generated. For example, it is now possible to produce transgenic animals (e.g., mice) that are capable, upon immunization, of producing a full repertoire of human antibodies in the absence of endogenous immunoglobulin production.

1 Alternatively, phage display technology can be used to produce human antibodies and antibody fragments in vitro, from immunoglobulin variable (V) domain gene repertoires from unimmunized donors. Phage display can be performed in a variety of formats. Several sources of V-gene segments can be used for phage display.

### Antibody Fragments

“Antibody fragments” comprise a portion of an antibody, preferably the antigen binding or variable region of the intact antibody. Examples of antibody fragments include Fab, Fab′, F(ab′)2, and Fv fragments; diabodies; linear antibodies; single chain antibody molecules; and multispecific antibodies formed from antibody fragments. Antibody fragments of particular interest are fragments that retain antigen-binding properties of the whole antibody, and are useful as inhibitors for practicing the invention.

Papain digestion of antibodies produces two identical antigen binding fragments, called “Fab” fragments, and a residual “Fc” fragment, a designation reflecting the ability to crystallize readily. Each Fab fragment is monovalent with respect to antigen binding, i.e., it has a single antigen binding site. Pepsin treatment of an antibody yields a single large F(ab′)₂ fragment which roughly corresponds to two disulfide linked Fab fragments having divalent antigen binding activity and is still capable of cross linking antigen. Fab′ fragments differ from Fab fragments by having additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen recognition binding site. This fragment consists of a dimer of one heavy and one light chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single chain Fv” abbreviated as “scFv” are antibody fragments that comprise the VH and VL antibody domains connected into a single polypeptide chain. Preferably, the scFv polypeptide further comprises a polypeptide linker between the VH and VL domains which enables the scFv to form the desired structure for antigen binding.

In certain circumstances there are advantages of using antibody fragments, rather than whole antibodies. The smaller size of the fragments allows for rapid clearance from the circulation.

Various techniques have been developed for the production of antibody fragments. Traditionally, these fragments were derived via proteolytic digestion of intact antibodies. However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)₂ fragments. According to another approach, F(ab′)₂ fragments can be isolated directly from recombinant host cell culture. Fab and F(ab′)₂ fragment with increased in vivo half-life comprising a salvage receptor binding epitope residues also may be used.

Other techniques for the production of antibody fragments will be apparent to the skilled practitioner. The antibody of choice is a single chain Fv fragment (scFv). Fv and scFv are the only species with intact combining sites that are devoid of constant regions; thus, they are suitable for reduced nonspecific binding during in vivo use. The antibody fragment may also be a “linear antibody”, which may be monospecific or bispecific. The inhibitor also may be a polypeptide or protein comprising an antibody or antibody fragment linked to another entity to form a fusion protein.

### Bispecific Antibodies

Bispecific antibodies are antibodies that have binding specificities for at least two different epitopes. Bispecific antibodies can be prepared as full length antibodies or antibody fragments (e.g., F(ab′)₂ bispecific antibodies).

Methods for making bispecific antibodies are known in the art. Traditional production of full length bispecific antibodies is based on the co-expression of two immunoglobulin heavy chain-light chain pairs, where the two chains have different specificities.

According to a different approach, antibody variable domains with the desired binding specificity (antibody-antigen combining sites) are fused to immunoglobulin constant domain sequences.

Bispecific antibodies include cross-linked or “heteroconjugate” antibodies. Heteroconjugate antibodies are composed of two covalently joined antibodies. It is contemplated that the antibodies may be prepared in vitro using known methods in synthetic protein chemistry, including those involving crosslinking agents. For example, one of the antibodies in the heteroconjugate can be coupled to avidin, the other to biotin. Heteroconjugate antibodies may be made using any convenient cross-linking methods. Suitable cross-linking agents and cross-linking techniques are well known in the art.

Techniques for generating bispecific antibodies from antibody fragments have also been described in the literature. For example, bispecific antibodies can be prepared using chemical linkage.

Recent progress has facilitated the direct recovery of Fab′-SH fragments from E. coli, which can be chemically coupled to form bispecific antibodies. Various techniques for making and isolating bispecific antibody fragments directly from recombinant cell culture have also been described.

The term “diabodies” refers to small antibody fragments prepared by constructing scFv fragments with short linkers (about 5 to 10 residues) between the VH and VL domains such that inter chain but not intra chain pairing of the V domains is achieved, resulting in a bivalent fragment, i.e., fragment having two antigen binding sites. Bispecific diabodies are heterodimers of two “crossover” scFv fragments in which the VH and VL domains of the two antibodies are present on different polypeptide chains.

According to an alternative “diabody” technology for making bispecific antibody fragments, the fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (scFv) dimers has also been reported.

Antibodies with more than two valencies are contemplated for use in the invention. For example, trispecific antibodies can be prepared.

### Multivalent Antibodies

A multivalent antibody may be internalized (and/or catabolized) faster than a bivalent antibody by a cell expressing an antigen to which the antibodies bind. Antibodies that may be used in the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. A preferred dimerization domain comprises an Fc region or a hinge region. In this scenario, the antibody will comprise an Fc region and three or more antigen binding sites amino-terminal to the Fc region. A preferred multivalent antibody comprises three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X₁)ₙ-VD2-(X₂)ₙ-Fc, wherein VD1 is a first variable domain, VD2 is a second variable domain, Fc is one polypeptide chain of an Fc region, X₁ and X₂ represent an amino acid or polypeptide, and n is 0 or 1. For instance, the polypeptide chain(s) may comprise: V_{H}-C_{H}1-flexible linker-V_{H}-C_{H}1-Fc region chain; or V_{H}-C_{H}1-V_{H}-C_{H}1-Fc region chain. A multivalent antibody preferably further comprises at least two (and preferably four) light chain variable domain polypeptides. A multivalent antibody may, for instance, comprise from about two to about eight light chain variable domain polypeptides. The light chain variable domain polypeptides contemplated here comprise a light chain variable domain and, optionally, further comprise a CL domain.

### Peptide and Peptide Mimetic Inhibitors

In another embodiment, the inhibitor of VEGF-D activity, VEGFR-3-mediated signaling or lymphangiogenesis is a peptide or peptide mimetic. The peptide or peptide mimetic may reduce receptor availability for native ligand binding.

As used herein, “peptide mimetic” and “peptidomimetic” are used interchangeably.

A peptide inhibitor is a peptide that binds specifically to a component of VEGF-C or VEGF-D activity, VEGFR-3-mediated signaling or lymphangiogenesis and inhibits or neutralizes the function of that component in the process of VEGF-C or VEGF-D activity, VEGFR-3-mediated signaling or lymphangiogenesis. Peptide inhibitors may be chemically synthesized using known peptide synthesis methodology or may be prepared and purified using recombinant technology. The preferred length of peptide inhibitors of VEGF-C or VEGF-D activity, VEGFR-3-mediated signaling or lymphangiogenesis is from about 6, 7, 8, 9 or 10 amino acid residues to about 100 amino acid residues. It is contemplated that longer peptides may prove useful. Peptide inhibitors may be identified without undue experimentation using well known techniques. In this regard, it is noted that techniques for screening peptide libraries for peptides that are capable of specifically binding to a polypeptide target are well known in the art.

For any of the foregoing peptides, one preferred variation involves peptides that have been modified to comprise an intramolecular bond between two non-adjacent amino acid residues of the primary sequence, thereby forming a cyclic peptide. For example, in one variation, the peptide comprises a pair of cysteine residues, such as amino- and carboxy-terminal cysteines, and the intramolecular bond comprises a disulfide bond between the cysteines. However, organic chemists and peptide chemists are capable of synthesizing intramolecular bonds between a wide variety of amino acids using conventional techniques.

### Nucleic Acid Molecules

### Antisense Molecules

In yet another embodiment, the inhibitor of VEGF-C or VEGF-D activity, VEGFR-3-mediated signaling or lymphangiogenesis is an antisense molecule that reduces transcription and/or translation of a component of VEGF-C or VEGF-D activity, VEGFR-3-mediated signaling or lymphangiogenesis, thereby reducing VEGF-C or VEGF-D activity, VEGFR-3-mediated signaling or lymphangiogenesis.

The antisense molecule comprises RNA or DNA prepared using antisense technology, where, for example, an antisense RNA or DNA molecule acts to block directly the translation of mRNA by hybridizing to targeted mRNA and preventing protein translation. Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. The antisense oligonucleotides thus may be used to reduce or block expression of a component of VEGF-C or VEGF-D activity, VEGFR-3-ediated signaling or lymphangiogenesis, and thus VEGF-C or VEGF-D activity, VEGFR-3-mediated signaling or lymphangiogenesis. Such oligonucleotides can also be delivered to cells such that the antisense RNA or DNA may be expressed in vivo to inhibit production of components of VEGF-C or VEGF-D activity, VEGFR-3-mediated signaling or lymphangiogenesis.

Inhibitors of VEGF-C or VEGF-D activity or signaling mediated by VEGFR-3, or lymphangiogenesis include antisense or sense oligonucleotides comprising a single-stranded nucleic acid sequence (either RNA or DNA) capable of binding to target mRNA (sense) or DNA (antisense) sequences. Such a fragment generally comprises about 10 to 40 nucleotides in length, preferably at least about 14 nucleotides, preferably from about 14 to 30 nucleotides.

Antisense or sense oligonucleotides further comprise oligonucleotides having modified sugar-phosphodiester backbones that are resistant to endogenous nucleases, or are covalently linked to other moieties that increases affinity of the oligonucleotide for a target nucleic acid sequence, or intercalating agents to modify binding specificities of the antisense or sense oligonucleotide for the target nucleotide sequence.

Small Interfering RNA (siRNA)

In one embodiment, it is envisaged that siRNA will inhibit VEGF-C or VEGF-D activity, VEGFR-3-mediated signaling or lymphangiogenesis. “siRNA” or “RNAi” are double-stranded RNA molecules, typically about 21 nucleotides in length, that are homologous to a gene or polynucleotide that encodes the target gene and interfere with the target gene's expression.

### Nucleic Acid Molecules in Triple-Helix Formation

In another embodiment, the inhibitor of VEGF-C or VEGF-D activity, VEGFR-3-mediated signaling or lymphangiogenesis comprises nucleic acid molecules in triple-helix formation. Nucleic acid molecules in triple-helix formation used to inhibit transcription should be single-stranded and composed of deoxynucleotides. A DNA oligonucleotide is designed to be complementary to a region of the gene involved in transcription. The base composition of these oligonucleotides is designed such that it promotes triple-helix formation via Hoogsteen base-pairing rules, which generally require sizeable stretches of purines or pyrimidines on one strand of a duplex.

### Ribozymes

In a related embodiment, the inhibitor of VEGF-C or VEGF-D activity, VEGFR-3-mediated signaling or lymphangiogenesis is a ribozyme that reduces transcription of a component of VEGF-C or VEGF-D activity or signaling mediated by VEGFR-3, or a lymphangiogenic component.

A “ribozyme” is an enzymatic RNA molecule capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques.

### Small Molecule Inhibitors

In a further embodiment, the inhibitor of VEGF-C or VEGF-D activity, VEGFR-3-mediated signaling or lymphangiogenesis is a small molecule.

A “small molecule” is defined herein to have a molecular weight below about 2000 daltons, and preferably below about 500 Daltons. Potential inhibitors of VEGF-C or VEGF-D activity, VEGFR-3-mediated signaling or lymphangiogenesis include small molecules that bind to the active site, the receptor binding site, or growth factor or other relevant binding site of components of VEGF-C or VEGF-D activity or VEGFR-3-mediated signaling, or lymphangiogenesis, thereby blocking the normal biological activity of VEGF-C or VEGF-D, VEGFR-3-mediated signaling or lymphangiogenesis. Examples of small molecules include, but are not limited to, synthetic non-peptidyl organic or inorganic compounds.

Small molecule inhibitors of VEGF-C or VEGF-D activity, VEGFR-3-mediated signaling or lymphangiogenesis may be identified without undue experimentation using known techniques and chemically synthesized using known methodology. In this regard, it is noted that techniques for screening organic molecule libraries for molecules that are capable of binding to a polypeptide target are known in the art.

### Inhibition of Receptor Availability for Ligand Binding

### Antibody Inhibitors

In one embodiment, the inhibitor of VEGF-C or VEGF-D activity, VEGFR-3-mediated signaling or lymphangiogenesis is an antibody. In a preferred embodiment, the inhibitor of VEGF-C or VEGF-D activity, VEGFR-3-mediated signaling or lymphangiogenesis is an anti-VEGFR-3 antibody that reduces VEGFR-3 availability for ligand binding.

Suitable antibodies for use in the methods of the invention and means for their production are disclosed in WO2000/021560 and WO1995/021868 and include a polyclonal or a monoclonal antibody that binds specifically to VEGFR-3 and blocks its signaling, a fragment of such an antibody, a chimeric antibody, a humanized antibody, and a bispecific antibody that binds specifically to VEGFR-3 and blocks its signaling and also binds to another antigen.

In a preferred embodiment, the antibody inhibitor is a humanized antibody. In another embodiment, the antibody inhibitor of VEGF-C or VEGF-D activity, VEGFR-3-mediated signaling or lymphangiogenesis comprises a Fab, Fab′, or F(ab′)₂ fragment, or a single chain Fv (scFv) fragment.

Persons skilled in the art will appreciate that in particular embodiments, the monoclonal antibody may comprise antibody 9D9F9, disclosed in WO2000/021560 or 2E11D11 disclosed in WO2003/006104. Alternatively monoclonal antibodies that specifically bind to VEGFR-3 and may be used according to the invention include antibodies MM0003-7G63, RM0003-5F63, C28G5, KLT9, ZMD.251, mF4-31C1 and hF4-3C5. A particularly preferred monoclonal antibody is hF4-3C5, a fully humanized antagonist antibody to human VEGFR-3.

In an alternative embodiment, the inhibitor may comprise a bispecific antibody, particularly a diabody, that binds specifically to and neutralizes each of VEGFR-3 and a second target. One example of such a diabody is that derived from antibodies hF4-3C5 and IMC-1121, which binds specifically to and neutralizes each of VEGFR-3 and VEGFR-2.

An inhibitor of VEGF-C or VEGF-D activity, VEGFR-3-mediated signaling or lymphangiogenesis according to the present invention also includes in one embodiment an antibody, as described above, that inhibits or neutralizes the receptor tyrosine kinase activity of VEGFR-3.

### Peptide and Peptide Mimetic Inhibitors

The person skilled in the art will appreciate that particular inhibitors of VEGF-C or VEGF-D activity, VEGFR-3-mediated signaling or lymphangiogenesis that can be employed in a particular embodiment of the present invention are disclosed in WO2000/021560, WO2001/052875, and WO2002/057299, which are incorporated herein by reference. In one embodiment, the inhibitor of VEGF-C or VEGF-D activity, VEGFR-3-mediated signaling or lymphangiogenesis comprises a peptide. Such a peptide to be used as an inhibitor of VEGF-C or VEGF-D activity, VEGFR-3-mediated signaling or lymphangiogenesis can be generated by random peptide synthesis, by recombinant means from random oligonucleotides, or a peptide may be selected from a phage display library, according to the disclosure of WO2002/057299 and WO2000/021560 and methods standard in the art. Such a peptide can be identified with the aid of the VEGFR-3 extracellular domain.

In a particular embodiment, the peptide inhibitor of VEGF-C or VEGF-D activity, VEGFR-3-mediated signaling or lymphangiogenesis comprises the amino acid sequence GYWX₁X₂X₃W (SEQ ID NO: 32), wherein X₁, X₂, and X₃ comprise amino acids and wherein the peptide binds VEGFR-3, according to WO2002/057299. In a related embodiment, the peptide inhibitor comprises the amino acid sequence GYWX₁X₂X₃WX₄ (SEQ ID NO: 33), wherein X₄ comprises an amino acid. In another embodiment, either of the preceding peptides may further comprise an amino- and carboxy-terminus cysteine residue. In a particular embodiment, the peptide comprises a cyclic peptide. In an alternative embodiment, the peptide comprises a peptide dimer that binds to VEGFR-3, and in a preferred form, the peptides comprising the dimer are the same, according to WO2002/057299.

In one embodiment, the peptidomimetic inhibitor is a monomeric monocyclic peptide inhibitor or dimeric bicyclic peptide inhibitor. Preferably, such peptidomimetic inhibitors are based on the peptide sequence of exposed loops of growth factor proteins, for example, loops 1, 2, and 3 of VEGF-D. In a preferred embodiment, the peptidomimetic inhibitor comprises any one of: CASELGKSTNTFC (SEQ ID NO: 43); CNEESLIC (SEQ ID NO: 44); or CISVPLTSVPC (SEQ ID NO: 45).

In one embodiment, the peptide mimetic inhibitor is prepared by the methods disclosed in WO2001/052875 and WO2002/057299. Peptides that may be used as inhibitors of VEGF-D activity, VEGFR-3-mediated signaling or lymphangiogenesis arc disclosed in WO2000/021560. Such peptides include a polypeptide comprising a fragment or analog of a vertebrate VEGF-C polypeptide, wherein the polypeptide and fragment or analog are capable of binding to VEGFR-3, but do not activate signaling, and a polypeptide comprising a fragment or analog of a vertebrate VEGF-C or VEGF-D polypeptide, wherein the polypeptide and fragment or analog are capable of binding to VEGFR-3, but do not activate signaling.

The person skilled in the art will appreciate that inhibitors of VEGF-D activity, VEGFR-3-mediated signaling or lymphangiogenesis inhibitors according to WO2002/057299 include peptides comprising the sequence Y₁GYWLTIWGY₂, (SEQ ID NO: 54) wherein Y₁, and Y₂ are amino acids. In one variation, the peptide is made cyclic by a bond between Y₁ and Y₂. In a specific preferred embodiment, the peptide comprises the sequence CGYWLTIWGC (SEQ ID NO: 42). Other peptide inhibitors comprise any of the following amino acid sequences: SGYWWDTWF (SEQ ID NO: 1), SCYWRDTWF (SEQ ID NO: 2), KVGWSSPDW (SEQ ID NO: 3), FVGWTKVLG (SEQ ID NO: 4), YSSSMRWRH (SEQ ID NO: 5), RWRGNAYPG (SEQ ID NO: 6), SAVFRGRWL (SEQ ID NO: 7), WFSASLRFR (SEQ ID NO: 8), WQLGRNWI (SEQ ID NO: 9), VEVQITQE (SEQ ID NO: 10), AGKASSLW (SEQ ID NO: 11), RALDSALA (SEQ ID NO: 12), YGFEAAW (SEQ ID NO: 13), YGFLWGM (SEQ ID NO: 14), SRWRILG (SEQ ID NO: 15), HKWQKRQ (SEQ ID NO: 16), MDPWGGW (SEQ ID NO: 17), RKVWDIR (SEQ ID NO: 18), VWDHGV (SEQ ID NO: 19), CWQLGRNWIC (SEQ ID NO: 20), CVEVQITQEC (SEQ ID NO: 21), CAGKASSLWC (SEQ ID NO: 22), CRALDSALAC (SEQ ID NO: 23), CYGFEAAWC (SEQ ID NO: 24), CYGFLWGMC (SEQ ID NO: 25), CSRWRILGC (SEQ ID NO: 26), CHKWQKRQC (SEQ ID NO: 27), CMDPWGGWC (SEQ ID NO: 28), CRKVWDIRC (SEQ ID NO: 29), CVWDHGVC (SEQ ID NO: 30), CGQMCTVWCSSGC (SEQ ID NO: 31), or conservative substitutions-variants thereof. Preferred peptides comprise these exact amino acid sequences, or sequences in which only one or only two conserved substitutions have been introduced. In another preferred variation, the peptides comprise amino- and carboxy-terminal cysteines, which permit formation of cyclic molecules and dimers and multimers. In yet another variation, peptide inhibitors include the amino acid sequence GYWX₁X₂X₃W (SEQ ID NO: 32), wherein X₁, X₂, and X₃ comprise amino acids, the amino acid sequence GYWX₁X₂X₃WX₄ (SEQ ID NO: 33), wherein X₄ comprises an amino acid. In still another variation, these peptides further comprise amino- and carboxy-terminal cysteine residues.

### Nucleic Acid Inhibitors

In a preferred embodiment, the invention envisages use of a VEGFR-3 antisense RNA, as disclosed in WO2000/021560, to inhibit the translation of VEGFR-3-encoding mRNA to eliminate or down-regulate levels of VEGFR-3. Similarly, siRNA or nucleic acids in triple helix formation could be used to reduce VEGFR-3 availability for ligand binding.

### Small Molecule Inhibitors

In a preferred embodiment, the small molecule is a small molecule inhibitor of receptor tyrosine kinase activity. In a more preferred embodiment, the small molecule comprises PTK787/ZK22854, AZP2171, ZK991, KRN633, MAZ51, sorafenib, sunitinib (SU11248), axitinib (AG013736), vandetanib (ZD6474), or 3-(indole-3-yl)-4-(3,4,5-trimethoxyphenyl)-1H-pyrrole-2,5-dione.

### Inhibition of Ligand Availability for Receptor Binding

### Antibody Inhibitors

According to one embodiment, inhibition of VEGF-C or VEGF-D activity, VEGFR-3-mediated signaling or lymphangiogenesis can be achieved using antibodies that specifically bind and neutralize ligands for VEGFR-3, that is, VEGF-C and/or VEGF-D. Antibodies similar to anti-VEGFR-3 antibodies described above are contemplated. Suitable antibodies and their means for production are disclosed in WO2000/021560. The person skilled in the art will appreciate that antibodies that bind specifically to VEGF-D and may be used according to the invention include monoclonal antibodies 2F8, 4A5 (also known as VD1), 4E10, 5F12, 4H4 and 3C10 disclosed in WO2000/037025. A particularly preferred antibody is 4A5, and in particular, a humanized version thereof. In another embodiment, the chimeric or humanized antibody comprises SEQ ID NO: 46 and SEQ ID NO: 47, or the antibody comprises any one of SEQ ID NOs: 48 to 50 and any one of SEQ ID NOs: 51 to 53, as disclosed in WO2005/087177. Alternatively monoclonal antibodies that may be used according to the invention include 28AT743.288.48, MM0007-7E79, RM0007-8C35, 78902, 78923, 78939, and 90409.

Similarly, monoclonal antibodies that bind VEGF-C may be employed. The anti-VEGF-C antibodies will specifically bind to human VEGF-C or a biologically active fragment thereof, e.g. the mature fully-processed form. Such binding will partially or fully block, neutralize, reduce or antagonize VEGF-C activity. Suitable examples of such antibodies include antibodies 103, MM0006-2E65 and 193208. Further examples of such antibodies are found in U.S. Pat. Nos. 7,208,582, 7,109,308.

One example of an anti-VEGF-C antibody is a monoclonal antibody that competitively inhibits the binding to VEGF-C of monoclonal anti-VEGF-C antibody 69D09 produced by hybridoma ATCC PTA-4095 or having the heavy and light chain amino acid sequences as follows:

```
Sequence of anti-VEGF-C antibody heavy chain
SEQ ID NO: 34
EVRLLESGGG LVQPGGSLRL SCAASGFTFR PRAMAWVRQA
                  10 20 30 40
PGKGLEWVSS ISAQGASAYY ADSVKGRFTI SRDNSKNTLY
                  50 60 70 80
LQMNSLRAED TAVYYCARDL SVSGFGPWGR GTMVTVSSAS
                  90 100 110 120
TKGPSVFPLA PSSKSTSGGT AALGCLVKDY FPEPVTVSWN
                  130 140 150 160
SGALTSGVHT FPAVLQSSGL YSLSSVVTVP SSSLGTQTYI
                  170 180 190 200
CNVNHKPSNT KVDKRVEPKS CDKTHTCPPC PAPELLGGPS
                  210 220 230 240
VFLFPPKPKD TLMISRTPEV TCVVVDVSHE DPEVKFNWYV
                  250 260 270 280
DGVEVHNAKT KPREEQYNST YRVVSVLTVL HQDWLNGKEY
                  290 300 310 320
KCKVSNKALP APIEKTISKA KGQPREPQVY TLPPSREEMT
                  330 340 350 360
KNQVSLTCLV KGFYPSDIAV EWESNGQPEN NYKTTPPVLD
                  370 380 390 400
SDGSFFLYSK LTVDKSRWQQ GNVFSCSVMH EALHNHYTQK
                  410 420 430 440
SLSLSPGK
              448
Sequence of anti-VEGF-C antibody light chain
SEQ ID NO: 35
SYELTQPPSS SGTPGQRVTI SCSGSSSNIG RHTVSWYQQV
                  10 20 30 40
PGTAPKLLIY SDDHRPSGVP DRFSASKSGT SASLTITGLQ
                  50 60 70 80
SEDEADYYCA AWDDSLNGPW VFGGGTKLTV LGQPKAAPSV
                  90 100 110 120
TLFPPSSEEL QANKATLVCL ISDFYPGAVT VAWKADSSPV
                  130 140 150 160
KAGVETTTPS KQSNNKYAAS SYLSLTPEQW KSHRSYSCQV
                  170 180 190 200
THEGSTVEKT VAPTECS
                  210 217
```

Another example of an anti-VEGF-C antibody is a monoclonal antibody that binds to the same epitope as the monoclonal anti-VEGF-C antibody 69D09 produced by hybridoma ATCC PTA-4095 or a monoclonal antibody having the heavy and light chain amino acid sequences shown above. In one embodiment, the anti-VEGF-C antibody is a fully-human anti-VEGF-C monoclonal antibody, including but not limited to 69D09 antibody or fragment thereof. The anti-VEGF-C antibody may be a humanized antibody.

Preferably, the anti-VEGF-C antibody is a human antibody produced by deposited hybridoma ATC PTA-4095 (also referred to herein as “VGX-100”) or having the heavy and light chain amino acid sequences shown above.

Alternatively, antibodies may bind proprotein convertases, enzymes responsible for processing VEGF-C and VEGF-D from their prepro-forms to their activated forms, and reduce, inhibit or neutralize such activity thereby limiting the amount of proteolytically processed ligand available for binding to VEGFR-3. Again, antibodies corresponding with anti-VEGFR-3 antibodies described above are envisaged. Such antibodies are disclosed in WO05/112971 and include neutralizing antibodies to inhibit the biological action of proprotein convertases.

### Peptide Inhibitors

Inhibitors of VEGF-C or VEGF-D activity, VEGFR-3-mediated signaling or lymphangiogenesis as used in the present invention include inhibitors of proprotein convertases. As noted, one class of inhibitor of proprotein convertases comprises antibodies. Another class of inhibitor of proprotein convertases includes peptide inhibitors.

Peptide inhibitors of proprotein convertases are disclosed in WO05/112971 and include prosegments of proprotein convertases, inhibitory variants of anti-trypsin and peptidyl haloalkylketone inhibitors.

Representative inhibitory prosegments of proprotein convertases include the inhibitory prosegments of PC5A (also known as PC6A), PC5B (also known as PC6B), PACE4, PCI (also known as PC3), PC2, PC4, PC7 and Furin. A representative inhibitory variant of anti-trypsin is α-1 antitrypsin Portland, an engineered variant of naturally occurring antitrypsin that inhibits multiple proprotein convertases. Representative peptidyl halomethyl ketone inhibitors include decanoyl-Arg-Val-Lys-Arg-chloromethylketone (Dec-RVKR-CMK), decanoyl-Phe-Ala-Lys-Arg-chloromethylketone (Dec-FAKR-CMK), decanoyl-Arg-Glu-Ile-Arg-chloromethylketone (Dec-REIR-CMK), and decanoyl-Arg-Glu-Lys-Arg-chloromethylketone (Dec-REKR-CMK). These inhibitors of proprotein convertases, such as Dec-RVKR-CMK or the inhibitory prosegments of proprotein convertases, can be used to block the activation of VEGF-C and VEGF-D and thereby inhibit VEGF-C or VEGF-D activity, VEGFR-3-mediated signaling or lymphangiogenesis induced by partially processed or fully processed VEGF-C or VEGF-D.

### Soluble Receptors

According to another embodiment, VEGF-C or VEGF-D activity, VEGFR-3-mediated signaling or lymphangiogenesis can be inhibited using soluble receptors that bind VEGFR-3 ligands. Soluble receptors capable of binding VEGF-C and VEGF-D, thereby inhibiting VEGF-C or VEGF-D activity or signaling via VEGFR-3, are disclosed in WO2000/023565, WO2000/021560 and WO2002/060950. Such inhibitors of VEGF-D activity, VEGFR-3-mediated signaling or lymphangiogenesis inhibitors include soluble VEGFR-2, VEGFR-3, NRP-1, and NRP-2.

### Nucleic Acid Inhibitors

In another embodiment of the invention, antisense oligonucleotides are used as inhibitors of proprotein convertases. The antisense oligonucleotides preferably inhibit expression of proprotein convertases by inhibiting transcription or translation of proprotein convertases. In a further embodiment, the antagonizing agent is small interfering RNAs (siRNA, also known as RNAi, RNA interference nucleic acids). Also contemplated are methods of inhibiting the target gene expression or target protein function utilizing ribozymes and triplex-forming nucleic acid molecules.

Similarly, in a related embodiment, antisense, siRNA and ribozyme inhibitors directed to VEGF-C and/or VEGF-D are included as inhibitors of VEGF-C or VEGF-D activity, VEGFR-3-mediated signaling or lymphangiogenesis exerting their effects by reducing transcription and/or translation of VEGF-C and VEGF-D.

### Peptide and Peptide Mimetic Inhibitors

According to one embodiment, the inhibitor to be used in the invention comprises a peptide that reduces the availability of ligand to bind to VEGFR-3. Such a peptide can be generated by random peptide synthesis, by recombinant means from random oligonucleotides, or a peptide may be selected from a phage display library by methods standard in the art. In a particular embodiment, the peptide will be derived from VEGFR-3 or VEGFR-2 and will bind specifically to VEGF-C or VEGF-D such that the ligand available for binding to native VEGFR-3 is reduced. Such a peptide may be identified with the aid of the VEGF-C or VEGF-D.

### Small Molecule Inhibitors

In one embodiment, the small molecule inhibitor is a small molecule inhibitor of a proprotein convertase. In a particular embodiment, the proprotein convertase is furin and the small molecule comprises B3 (CCG8294, naphthofluorescein disodium) or a derivative of 2,5-dideoxystreptamine.

### Antibody Inhibitors Affecting Ligand-Receptor Complex

In one embodiment, the invention includes use of bispecific antibodies, as described above, as inhibitors of VEGF-C or VEGF-D activity, VEGFR-3-mediated signaling or lymphangiogenesis, specifically inhibiting ligand-receptor complexes.

Suitable antibodies and their means for production are disclosed in WO2000/021560 and include a bispecific antibody that binds specifically to an epitope or epitopes derived from a VEGFR-3 (VEGFR-3 ligand) complex (receptor-ligand complex) and blocks VEGFR-3 signaling.

### Inhibition of Co-Receptor Interaction

### Antibody Inhibitors Affecting Co-Receptors of VEGFR-3

In a further embodiment, inhibitors of VEGF-C or VEGF-D activity, VEGFR-3-mediated signaling or lymphangiogenesis include antibodies, as described above, that bind specifically to and reduce, inhibit or neutralize co-receptor binding to VEGFR-3. Such antibodies may be directed to a co-receptor, a ligand-co-receptor binary complex, a co-receptor-receptor binary complex, or a ligand-co-receptor-receptor thernary complex. Co-receptors include NRP-1 and NRP-2. The person skilled in the art will understand that monoclonal antibodies that specifically bind NRP-1 or NRP-2 and may be used according to the invention include antibodies 1B3, 3G6-2C5, AD5-17F6, 446915, 446921, 130603, 130604, 96009, 3B8, 54, 257103, 257107, A-12, and C-9. Alternatively, a bispecific antibody which specifically binds to NRP-2 receptor and a VEGF-C polypeptide, as disclosed in WO2003/029814, may be used according to the invention.

### Peptide Inhibitors Affecting Co-Receptors of VEGFR-3

In another embodiment, a peptide inhibitor comprising a peptide dimer may target one or more receptors and/or co-receptors. Co-receptors include NRP-1 and NRP-2. As disclosed in WO2002/057299, in a particular embodiment, the peptide dimer comprises one peptide that binds VEGFR-3 and a second peptide that binds to any one of VEGFR-1, VEGFR-2, NRP-1, or NRP-2.

### Small Molecule and Nucleic Acid Inhibitors Affecting Co-Receptors of VEGFR-3

According to the present invention, it is also envisaged that small molecules, antisense molecules, siRNA and ribozymes, as described above, can be utilized as inhibitors of VEGF-D activity, VEGFR-3-mediated signaling or lymphangiogenesis by targeting co-receptors that interact with VEGFR-3. Such co-receptors include NRP-1 and NRP-2.

### Inhibition of Downstream Signaling

Alternatively, an inhibitor of VEGF-C or VEGF-D activity, VEGFR-3-mediated signaling or lymphangiogenesis according to any of the foregoing descriptions may disrupt downstream intracellular VEGFR-3 signaling, as disclosed in WO2000/021560.

### Pharmaceutically Acceptable Carriers

Suitable ophthalmic carriers are known to those skilled in the art and all such conventional carriers may be employed in the present invention. Exemplary compounds incorporated to facilitate and expedite transdermal delivery of topical compositions into ocular or adnexal tissues include, but are not limited to, alcohol (ethanol, propanol, and nonanol), fatty alcohol (lauryl alcohol), fatty acid (valeric acid, caproic acid and capric acid), fatty acid ester (isopropyl myristate and isopropyl n-hexanoate), alkyl ester (ethyl acetate and butyl acetate), polyol (propylene glycol, propanedione and hexanetriol), sulfoxide (dimethylsulfoxide and decylmethylsulfoxide), amide (urea, dimethylacetamide and pyrrolidone derivatives), surfactant (sodium lauryl sulfate, cetyltrimethylannmonium bromide, polaxamers, spans, tweens, bile salts and lecithin), terpene (d-limonene, alphaterpencol, 1,8-cincole and menthone), and alkanone (N-heptane and N-nonane). Moreover, topically-administered compositions comprise surface adhesion molecule modulating agents including, but not limited to, a cadherin antagonist, a selectin antagonist, and an integrin antagonist. Thus, a particular carrier may take the form of a sterile, ophthalmic ointment, cream, gel, solution, or dispersion. Also including as suitable ophthalmic carriers are slow release polymers, e.g., “Ocusert” polymers, “Hydron” polymers, etc.

Stabilizers may also be used such as, for example, chelating agents, e.g., EDTA. Antioxidants may also be used, e.g., sodium bisulfite, sodium thiosulfite, 8-hydroxy quinoline or ascorbic acid. Sterility typically will be maintained by conventional ophthalmic preservatives, e.g., chiorbutanol, benzalkonium chloride, cetylpyridium chloride, phenyl mercuric salts, thimerosal, etc., for aqueous formulations, and used in amounts which are nontoxic and which generally vary from about 0.001 to about 0.1% by weight of the aqueous solution. Conventional preservatives for ointments include methyl and propyl parabens. Typical ointment bases include white petrolatum and mineral oil or liquid petrolatum. However, preserved aqueous carriers are preferred. Solutions may be manually delivered to the eye in suitable dosage form, e.g., eye drops, or delivered by suitable microdrop or spray apparatus typically affording a metered dose of medicament. Examples of suitable ophthalmic carriers include sterile, substantially isotonic, aqueous solutions containing minor amounts, i.e., less than about 5% by weight hydroxypropylmethylcellulose, polyvinyl alcohol, carboxymethylcellulose, hydroxyethylcelullose, glycerine and EDTA. The solutions are preferably maintained at substantially neutral pH and isotonic with appropriate amounts of conventional buffers, e.g., phosphate, borate, acetate, tris.

All publications and patents mentioned herein are hereby incorporated by reference in their entirety as if each individual publication or patent was specifically and individually indicated to be incorporated by reference. In case of conflict, the present application, including any definitions herein, will control.

### EXAMPLES

### Materials and Methods

### Experimental Dry Eye Murine Model

Eight to ten week-old female C57BLI6 mice (Charles River Laboratory, Wilmington, MA) were used in accordance with the standards in the ARVO Statement for the Use of Animals in Ophthalmic and Vision Research. The research protocol was approved by the Schepens Eye Research Institute Animal Care and Use Committee. Dry eye was induced in murine eyes using a Controlled Environment Chamber (CEC) which exposes the mice to high-flow desiccated air. To achieve maximum ocular surface dryness, the conditions in CEC were supplemented with topical application of 1% atropine sulfate (Falcon Pharma, Fort Worth, Texas) twice for the first 48 hours and subcutaneous injections of 0.1 ml of 5 mg/ml of scopolamine hydrobromide (Sigma-Aldrich, St. Louis, MO) three times a day, for the entire duration of the experiment.

RNA Isolation and Molecular analysis using Real Time Polymerase Chain Reaction

Five mice (10 eyes) were included in each group. Two corneas were pooled together to equal as one sample and stored at −80′° C. in Trizol (Invitrogen, Carlsbad, California; catalog No.15596026) until future use. Total RNA was isolated from these corneas using the RNeasy microkit (Qiagen, Valencia, CA; catalog No. 74004). Equal amounts of RNA were used to synthesize cDNA using SuperScript™ III Reverse Trancriptase (Invitrogen, Carlsbad, CA; catalog No.18080) according to the manufacturer's recommendations. Real-Time PCR was performed using FAM-MGB dye labeled predesigned primers (Applied Biosystem, Foster City, CA) for GAPDH (assay ID.Mm999999 15_gl), VEGF-A (Mm00437304_ml), VEGF-C(Mrn00437313_ml), VEGF-D (Mm00438965_ml), VEGFR-2 (Mm00440099_ml), VEGFR-3 (Mm00433337_ml). 2.5 μl of cDNA was loaded in each well and assays were performed in duplicate. The GAPDH gene was used as the endogenous reference for each reaction. The results were normalized by the cycle threshold (CT) of GAPDH and the relative mRNA level in the normal mice was used as the normalized control.

### Immunohistochemistry

The following primary antibodies were used for immunohistochemical staining: rat anti-mouse CD11b-FITC for monocytes/macrophages (BD Pharmingen, San Diego, California, 1:100), goat anti-mouse CD31 FITC as pan-endothelial marker (Santa Cruz Biotechnology, Santa Cruz, California, 1:100) and purified rabbit anti-mouse LYVE-1 as lymphatic endothelial marker (Abeam, MA, USA, 1:400). Respective isotypes were used as negative controls.Rhodamine conjugated goat anti-rabbit (BD Pharmingen, San Diego, California, 1:100) was the secondary antibody used.

Freshly excised corneas were washed in PBS, fixed in acetone for 15 minutes and then double stained with CD31 and LYVE-1 as described previously. To analyze infiltration of CD11b⁺/LYVE-1 cells, corneas from three mice from each group were taken and cells were counted in 5-6 areas in the periphery (0.5 μm area from the limbus) of each cornea in a masked fashion, using epifluorescence microscope (model E800; Nikon, Melville, NY) at 40× magnification. The mean number of cells was obtained by averaging the total number of cells in all the areas studied and the result was expressed as the number of positive cells per mm2.

### Morphometry of Lymphangiogenesis in the Cornea

Morphology of lymphatics was analyzed using an automated image analysis program written with Matlab (The Mathworks, Inc., Natick, MA). Lymphatics were isolated from digitized images with this program using standard computer vision techniques for image segmentation, including background isolation and subtraction, edge detection, and k-means clustering. This segmentation process generated binary images in which lymphatic vessels are represented by 1s and all other image content is represented by Os. The resultant isolated lymphatic vessels were analyzed morphologically using two metrices, Lymphatic Area (LA) and Lymphatic Caliber (LC). LA represents the total surface area of the lymphatic vessels when projected into the plane of the image. LC is a summary measure of the diameters of the lymphatic vessels present. LC was measured using a computational technique that generates the largest diameter circle centered at each pixel inside a lymphatic vessel. The mean value across all pixels within lymphatic vessels was taken as an estimate of the mean LC for a given image.

### Flow Cytometry

Draining LNs from DED (day 10) and normal mice were collected. Single cell suspension of LN cells was stained with the anti-CD11b-FITC and anti-lab (MHC-II)-PE. Stained LN cells were then analyzed on an EPICS XL flow cytometer (Beckman Coulter). All the antibodies with their matched isotype controls were purchased from eBioscience.

Studies involving inhibition of corneal neo-lymphangiogenesis using an anti-VEGF-C antibody (Example 5 onwards)

Anti-VEGF-C antibodies (VGX-100; Vegenics Limited, Australia) were administered intraperitonealy daily from day 1 to day 10 to DED mice. Mice were assessed clinically using corneal fluorescent staining. Tissues from cornea, conjunctiva and draining lymph nodes were examined for cellular and molecular pathological changes. In vivo blockade of VEGF-C suppresses corneallymphangiogenesis and ameliorates clinical signs of DED.

### Statistical Analysis

A two-tailed Student's t-test was performed and P-values less than 0.05 were deemed statistically significant. Results are presented as the mean±SEM of at least three experiments.

### Example 1: Demonstration and Quantification of Lymphatics in Dry Eye Corneas

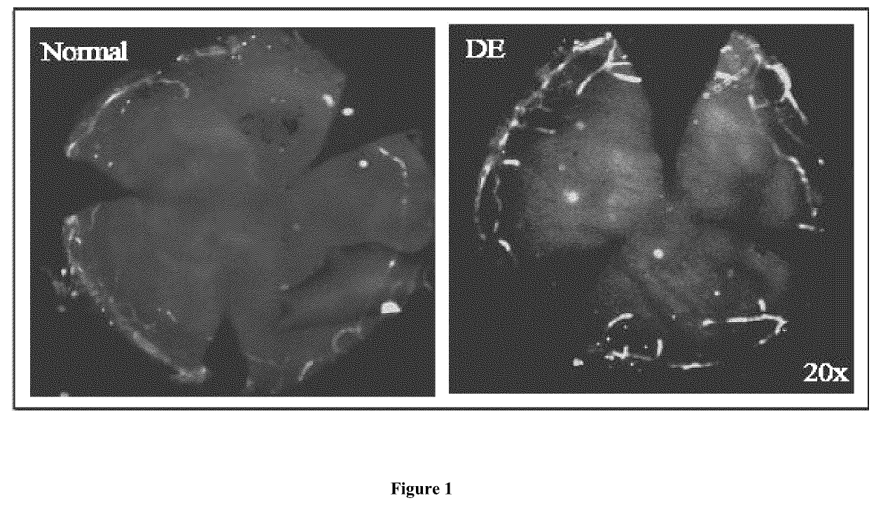

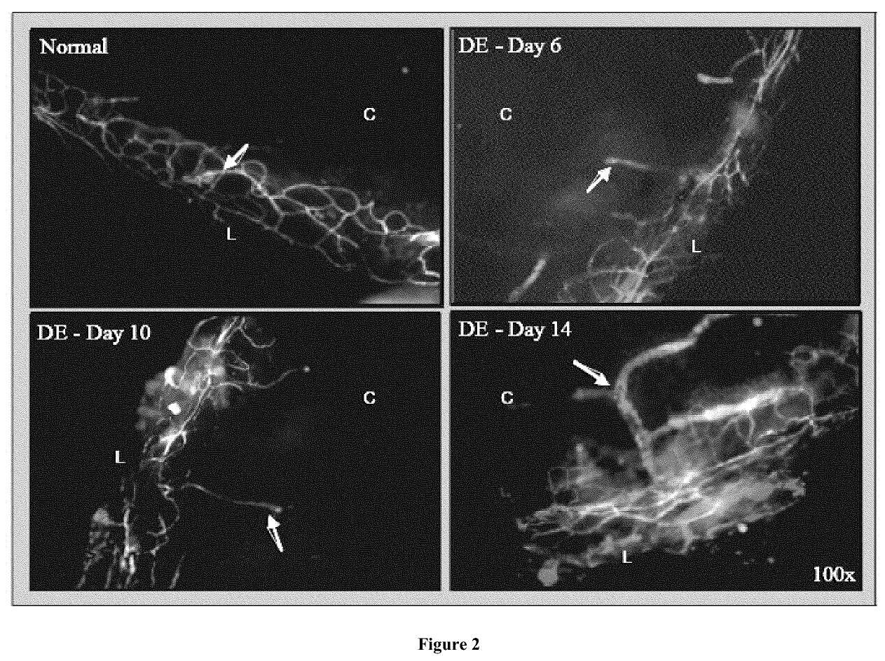

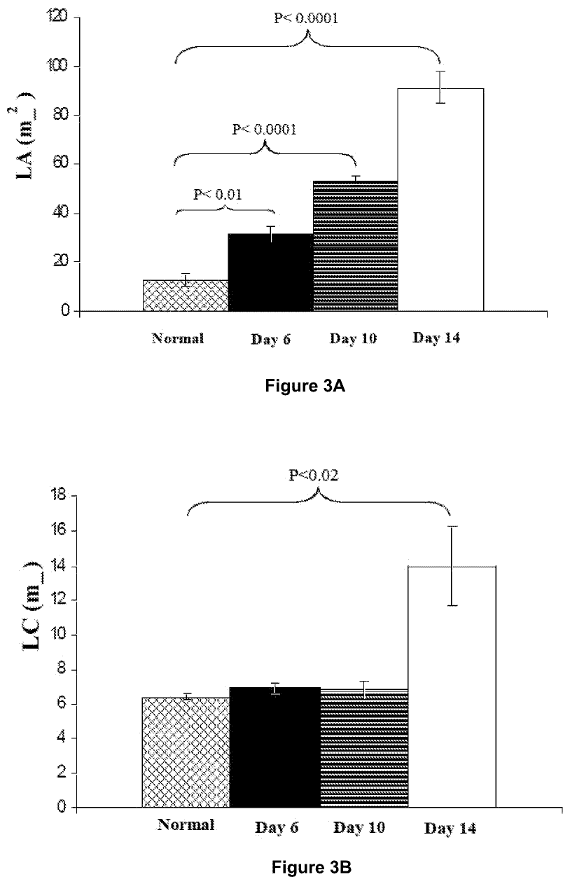

To determine whether DED induces growth of lymphatics into the cornea, and whether lymphatic growth is paralleled by growth of blood vessels, corneal whole mounts were double stained for CD31 (pan-endothelial marker) and LYVE-1 (lymphatic vascular endothelial marker) at days 0, 6, 10 and 14 and quantified for lymphangiogenesis. Blood vessels were identified as CD31^{hi}/LYVE-1- and lymph vessels were identified as CD31^{lo}/LYVE-1^{hi}. A significant increase in lymphatic area LA is seen in DED mice (FIG. 1b). Morphometric analysis revealed small buds of lymphatic vessels arising from the limbal vascular arcade at an early time point (day 6), which increased in caliber (LC) and area (LA), and advanced towards the center of the cornea with DED progression (FIGS. 1 and 2). A significant increase in LA (FIG. 3a) was seen as early as day 6 (P<0.01) which continued until day 14 (P<0.0001). However, LC (FIG. 3b) was significantly increased from the normal only by day 14 (P<0.02). Remarkably, these lymphatics were not accompanied by growth of blood vessels at any given time point.

### Example 2: Expression Levels of Different VEGF's and VEGFR's in Dry Eye Corneas

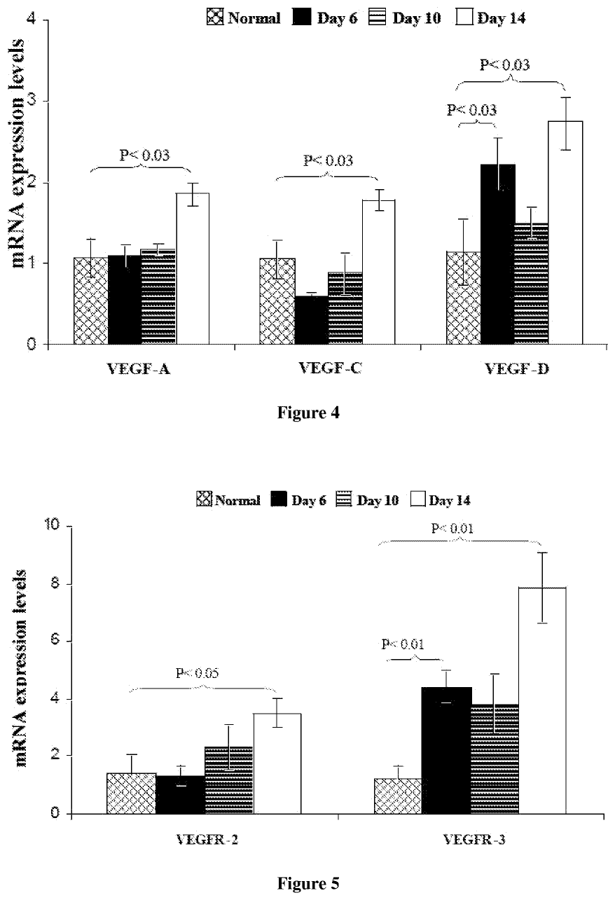

The development of lymphatic vessels is regulated by factors common to both hemangiogenesis and lymphangiogenesis. VEGF-C and VEGF-D are the classic lymphangiogenic factors and act by binding to their receptors VEGFR-2 and VEGFR-3, which are expressed on lymphatic endothelial cells. To determine the molecular mechanisms of lymphangiogenesis in DED, expression of different vascular endothelial growth factors and their receptors were quantified at indicated time points in the cornea using real time PCR. Amongst the VEGF species (FIG. 4), lymphangiogenic specific VEGF-D was not only the earliest to increase at day 6 (−2 folds; P<0.03) but also showed the maximum increase in expression at day 14 (−3 folds; P<0.03). Significant increased transcript expression of VEGF-A and VEGF-C was seen only by day 14 (P<0.03 for both). Similarly levels of lymphangiogenic specific VEGFR-3 were first to show a significant increase at day 6 (−4 folds; P<0.01) and continued to rise until day 14 (−8 folds; P<0.01). Though an overall trend toward increased expression was noticed with VEGFR-2 (primarily specific for blood vessel growth), significant increase (P<0.05) was appreciated only by day 14 (FIG. 5).

### Example 3: Enumeration of CD11b/LYVE-1 Positive Cells in Dry Eye Corneas

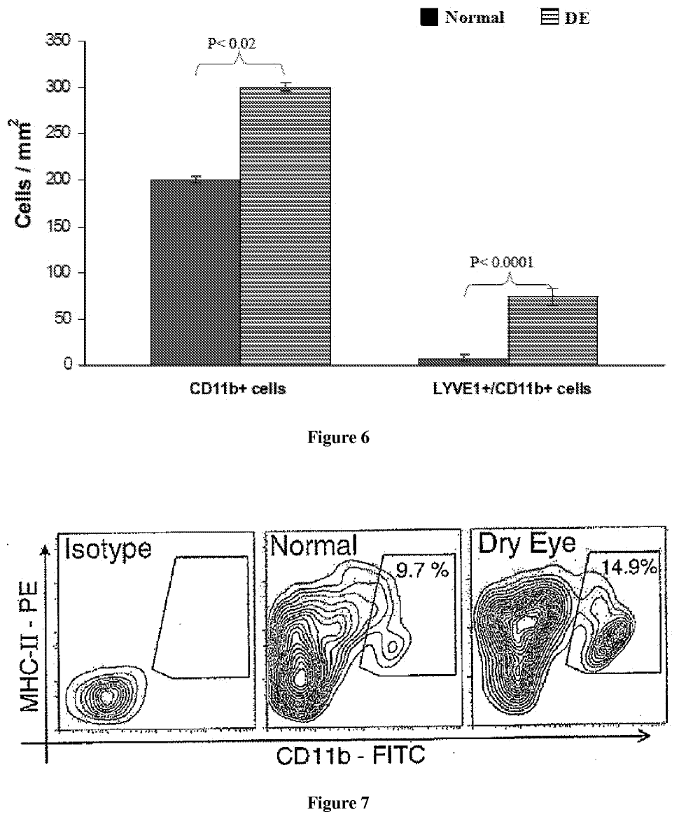

The normal cornea has a resident population of bone marrow-derived CD11b⁺ monocyticmacrophage-lineage cells and the development of DED increases the number of CD11b⁺ cells in the cornea. The role of macrophages in inflammatory lymphangiogenesis is well established. These CD11b⁺ macrophages may also express various lymphatic endothelial markers, such as LYVE-1. To see what proportion of these CD11b⁺ cells had lymphangiogenic potential, whole mount corneal tissues were double stained with CD11b and LYVE-1 at day 14. There was a significant increase in the number of both CD11b⁺ (P<0.02) and CD11b⁺/LYVE-1⁺ (P<0.0001) cells in dry eye as compared to normal corneas (FIG. 6). In DED, about 25% of the CD11b⁺ cells were positive for LYVE-1 where as only 4% of the CD11b⁺ cells were positive for LYVE-1 in the normal corneas.

### Example 4: Role of APC Homing

It was next investigated whether corneal lymphangiogenesis in DED is associated with the increased homing of APC in the draining LN. Using flow cytometry, the frequencies of mature APC (MHC-II+CD11b⁺) in the draining LN of normal and DED mice were analysed (FIG. 7). Data showed a significant increase in the frequency of MHC-II+CD11b⁺ APC in the LN cells of DED mice compared to those in the LN of normal mice (Range: 14.9-19.5% vs. 10-13.5%, p<0.05).

### Example 5: Effect of In Vivo Blockade of Pro-Lymphangiogenesic VEGF-C on Dry Eye Disease

Dry eye was induced in murine eyes as described in the materials and methods.

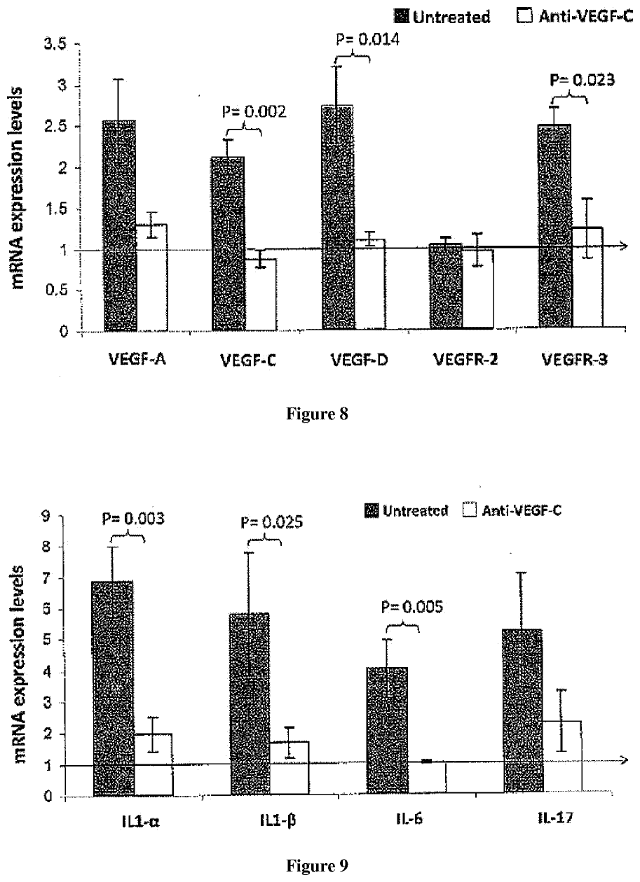

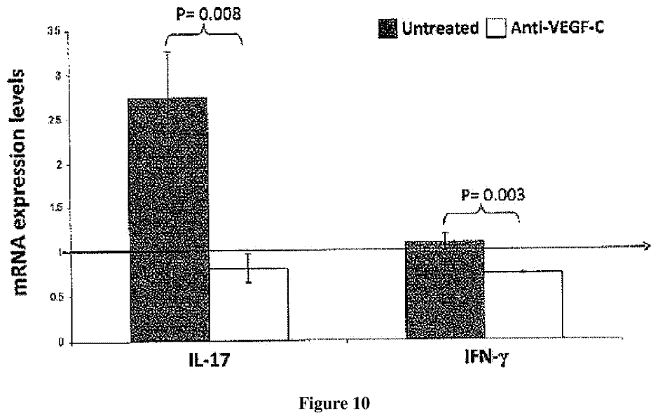

Real time PCR was performed to quantify expression of different VEGF growth factors (VEGF-A, VEGF-C, VEGF-D) and their receptors (VEGFR-2, VEGFR-3) in the cornea at days 6, 10 and 14 (FIG. 8) and to determine the levels of proinflammatory cytokines. IL-1α, IL-1β, IL-6, IL-17 in the conjunctiva showed significantly decreased expression in anti-VEGF-C treated DED mice as compared to those of untreated DED mice (FIG. 9). Draining lymph nodes of anti-VEGF-C treated DED mice showed significantly decreased induction of T-cell mediated autoimmune response compared untreated DED mice as determined by Real-time PCR analysis for IL-17 (Th17 cells) and IFN-γ (Th1 cells) (FIG. 10).

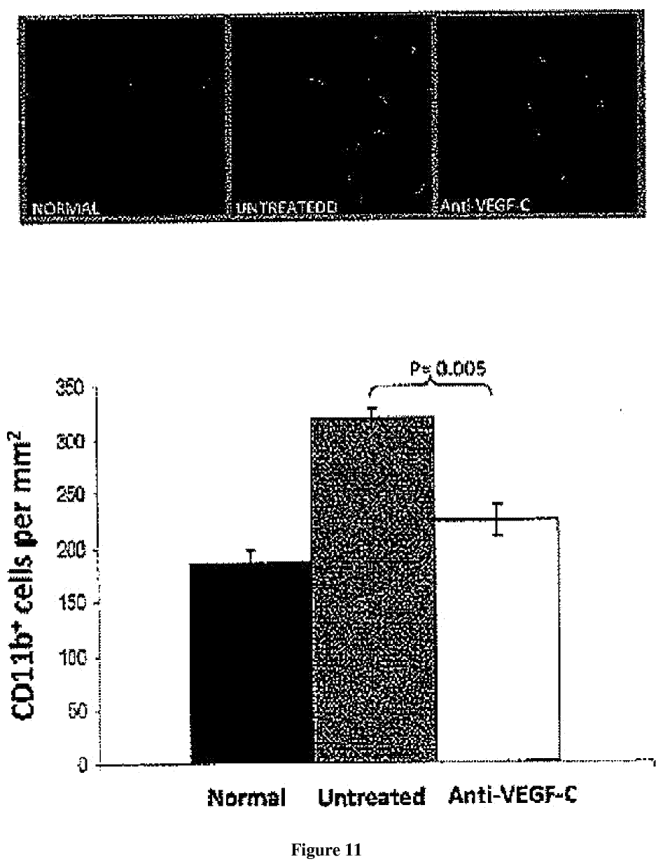

Enumeration of CD11b⁺/LYVE-1⁺ monocytic cells was done in the DED corneas at day 14 as described previously (FIG. 11). Treatment with anti-VEGF-C antibodies significantly decreased infiltration of CD11b⁺ cells (30%) in the DED corneas.

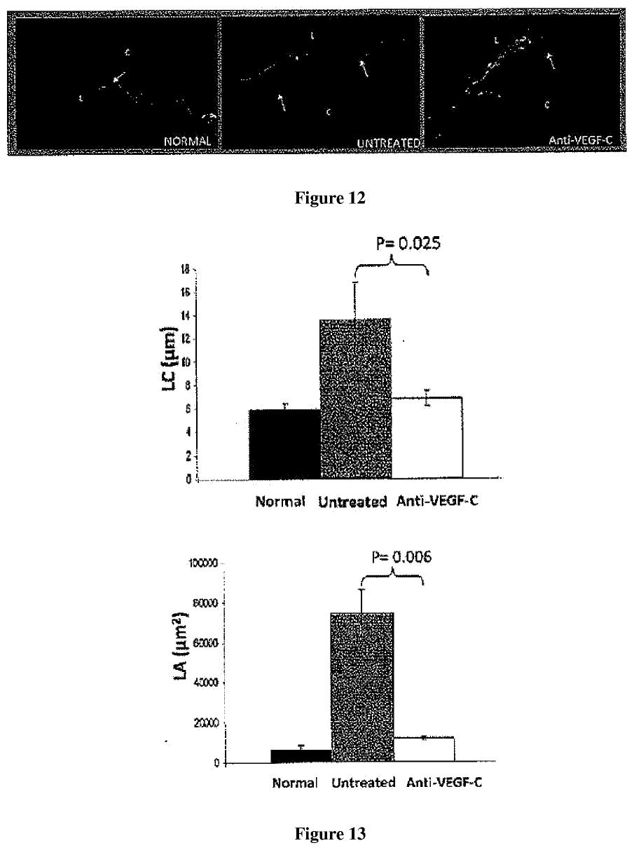

To determine whether DED induces growth of lymphatics into the cornea, and whether lymphatic growth is paralleled by growth of blood vessels, corneal whole mounts were double stained for CD31 (pan-endothelial marker) and LYVE-1 (lymphatic vascular endothelial marker) at days 0, 6, 10 and 14 and quantified for lymphangiogenesis as described previously. Lymphatics were seen growing toward the center of DED corneas (FIG. 12). Morphometric analysis showed significant increase in both lymphatic area (P<0.0001) and lymphatic caliber (P<0.02) at day 14 of disease (FIG. 13). These lymphatics were not accompanied by any new blood vessels. Lymphangiogenic specific VEGF-D and VEGFR-3 were the earliest to increase at day 6 followed by increase in VEGF-C, VEGF-A and VEGFR-2. Increased recruitment of CD11b⁺/LYVE-1⁺ monocytic cells to the cornea was also seen with disease.

These results demonstrate that low-grade inflammation associated with dry eye is an inducer of lymphangiogenesis without accompanied hemangiogenesis.

Clinical Relevance: Demonstration of selective lymphatic growth into dry eye corneas

provides an important mechanistic link to adaptive (T cell-meditated) immunity by delineating how corneal antigen trafficking can occur to the lymphoid tissues.

Dry eye disease (DED) once thought to be solely due to deficiency of tears, is increasingly being recognized as an immune-mediated disorder) DED affects many millions of people with a wide spectrum of seminal features ranging from mild ocular discomfort to sight-threatening corneal complications such as persistent epithelial defects and sterile stromal ulceration) In the United States alone, more than 3.2 million women and 1.6 million men above the age of 50 years are affected by this potentially disabling disease adversely impacting the vision-related quality of life.

Clinically significant DED is associated with ocular surface inflammation, although the precise immunopathogenesis is not known. There is strong evidence regarding T cell involvement in the pathogenesis of DED in both animal models and humans. Recently, we illustrated T cell activation in the regional lymph nodes of dry eye mice, coincident with acquisition of specific chemokine markers which help in the homing of T cells to the inflamed ocular surface. Further we demonstrated induction of autoimmunity in the draining lymph nodes of dry eye mice due to impaired Treg function and generation of pathogenic Th 17 cells. These Th 17 cells were found to be resistant to Treg mediated suppression, leading to unrestrained generation of pathogenic T cells and sustained ocular surface inflammation. Accordingly, much of the work to date has focused on understanding immunological phenomena occurring in the lymphoid compartment and the effector responses thereby generated, leaving unanswered the question as to how naive T cells in the draining lymph nodes get primed to the ocular surface antigen(s) that drive immunity in DED.

The draining lymph nodes are critical sites for induction of immunity and their role in generation of alloimmunity has been well established in corneal transplantation. The enhanced survival rate of corneal transplants in mice with excised cervical lymph nodes implicates the importance of functional flow of antigen presenting cells (APCs) from the ocular surface to the to the draining lymphoid tissue as a necessary component of alloimmunity and graft rejection. However, little is known about the pathway that allows trafficking of corneal APCs to the draining lymph nodes where they prime naive T cells to corneal antigens and generate autoimmune responses in dry eye.

Emphasis is now being given to the importance of pathological angiogenesis (hem- and lymphangiogenesis) in various corneal diseases such as different forms of keratitis, chemical burns, graft vs host disease etc., but to date there is no data regarding corneal angiogenesis in DED. A plausible reason could be that most of the above mentioned conditions except DED are accompanied by in-growth of clinically visible blood vessels into the cornea. Traditionally it has been thought that lymphatics and blood vessels which serve as afferent and efferent arms of the immune response respectively are always coexistent in pathological states. The present work provides the first evidence for selective lymphangiogenesis occurring in DED cornea using a murine model. Herein, we attempt to determine the growth of lymphatic vessels into the cornea with the progression of DED, discuss the pathophysiologic implications of corneal lymphangiogenesis in dry eye and the potential of antilymphangiogenic therapy for ameliorating DED.

### DISCUSSION

Lymphangiogenesis in the postnatal period is primarily a response to inflammation and is seen in various pathological states as diverse as tumor metastasis, wound healing and transplantation. Lymphatics play an important role in generating immuno-inflammatory responses by directing the antigen bearing immunocytes (e.g. dendritic cells) from the periphery to the draining lymph nodes where T cells are primed and expanded. The normal human cornea is avascular, thus suppressing the afferent lymphatic and efferent vascular arms of the immune cycle. Inflammation however negates this “immune” and “angiogenic” privileged state of the cornea and gives it the potential to mount an immune response.

Angiogenesis in the cornea is now extensively being studied in various pathological models such as transplantation. Whereas corneal blood vessels have long been thought to be an important risk factor for immune rejection in corneal transplantation, it is only recently after unveiling of new lymphatic specific markers, that the significance of lymphangiogenesis in corneal alloimmunity has been characterized. Despite recognizing the role of inflammatory angiogenesis in the eye, little has hitherto been studied regarding angiogenic mechanisms in DED. Desiccating stress in DED initiates an immune-based inflammatory response that is sustained by the ongoing interplay between the ocular surface and various pathogenic immune cells, primarily the CD4⁺ T cells in the conjunctiva and CD11b+ monocytic cells in the cornea. Desiccating stress induces secretion of inflammatory cytokines, especially interleukin (IL)-1, tumor necrosis factor-a, and IL-6 by ocular surface tissues, which facilitate the activation and migration of resident APCs toward the regional draining LN. Our data on frequencies of mature APC in the LN also suggest increased trafficking of mature APC in the LN of DED mice (FIG. 7). In the LN, these APCs stimulate naive T cells, leading to the expansion of IL-17-secreting Th 17 cells and interferon (IFN)-γ-secreting Th1 cells. Once these effectors are generated in the LN, they migrate to the ocular surface and secrete effector cytokines. Recent work has provided evidence for the induction of T cell mediated autoimmune responses in the regional lymph nodes of DED mice. But what has remained unanswered is how corneal APCs can traffic to the draining lymphoid compartment in order to initiate the immune cycle in DED.

Interestingly, to date there has been no published data on this important facet of immunity in DED. The data presented herein clearly demonstrates the development of lymphatic vessels in the setting of the dry eye state. These lymphatic vessels increase both in caliber and area while advancing toward the corneal center with progression of dry eye. Remarkably, these lymphatic vessels are not accompanied by growth of blood vessels. Various spatio-temporal studies examining relation between new blood and lymphatic vessels have led to the belief that a preexisting blood vascular bed is necessary to guide lymphangiogenesis. The current study refutes the general perception of wound healing models in skin where growth of lymphatic vessels follows that of blood vessels by several days. This is also in contrast to other robust models of corneal inflammation where there is either parallel outgrowth of blood and lymphatic vessels or the blood vessels are precedent over the lymphatics. This provides the first evidence of selective ‘natural’ (non pharmacologically induced) lymphangiogeneis in a disease model that is dissociated from hemangiogenesis.

Lymphangiogenesis is mediated primarily by the interaction of growth factors VEGF-C and VEGF-D on VEGFR-2 and VEGFR-3. VEGF-A also contributes, albeit indirectly, to lymphangiogenesis by recruiting VEGF-C and VEGF-D secreting macrophages. In the present study, dry eye induction led to the up-regulation of all the VEGF growth factors and their receptors. Though the rise in levels of VEGF-A, VEGF-C and VEGFR-2 occurred at later time points (day 14), it is noteworthy, that VEGF-D and VEGFR-3 (which are both largely specific to lymphangiogenesis) increased as early as day 6 of disease. The functional relevance of the early rise of VEGF-D is highlighted in a recent study where VEGF-D, via its action on VEGFR-3, was shown to be a critical modifier of VEGF-C driven early sprouting and migration of lymphatic endothelial cells. Macrophages also seem to play a crucial role in lymphangiogenesis. Under normal physiological conditions, all ocular tissues except the central cornea are rich in bone marrow derived LYVE-1⁺ macrophages which may serve as precursor cells for de novo formation of lymphatics. In the present study, we noticed significantly increased number of CD11b⁺/LYVE-1⁺ cells in the peripheral corneas after exposure to desiccating stress, suggesting that either these cells infiltrate into or multiply from pre-existing CD11b⁺/LYVE-1⁺ cells in the cornea, and contribute to lymphangiogenesis. Alternatively, there is a possibility of upregulation of LYVE-1 in the previous CD11b⁺/LYVE-1′ cells.

In summary, presented herein is novel evidence for the selective growth of lymphatic (but not blood) vessels in dry eye disease providing new insights into the pathophysiology of the disease. The findings suggest that these newly formed corneal lymphatics may serve as potential conduits for migration of corneal APCs to lymphoid tissues where they generate autoreactive Th 17 and Th1 cells in DED. This study not only provides a link between ocular surface inflammation and the generation of T cell mediated immunity in the lymphoid compartment, but also offers an example of how lymphangiogenesis and hemangiogenesis can be ‘naturally’ dissociated in a pathological state. The severing of the ‘eye-lymphatic axis’ in other immune-mediated conditions, such as transplant rejection, has been shown to hold promise as a strategy of suppressing alloimmunity without inhibiting needed innate host defense mechanisms. Similarly, a strategy targeting prolymphangiogenic factors such as VEGF-C or VEGF-D may prove effective in ameliorating dry eye disease.

### Example 7: Blockade of Prolymphangiogenic VEGF-C Suppresses Dry Eye Disease

Effect of in vivo blockade of pro-lymphangiogenic VEGF-C on Dry Eye Disease Rationale: Dry eye disease (DED) is an immune-mediated disorder whose precise pathogenesis remains largely unknown. While it has been clearly established that in DED generation of pathogenic CD4⁺ T cells (Th1/Th17) primarily occur in the draining lymph nodes, the mechanisms of trafficking of corneal antigen presenting cells (APC) to lymphoid tissues where they activate and expand pathogenic CD4⁺ T cell subsets, were still not well understood prior to the invention described herein. The present invention provides evidence for the selective growth of lymphatic (but not blood) vessels in DED cornea. Data shows a significant increase in both caliber and extent of lymphatics in DED corneas which was also confirmed using real-time PCR by showing a highly significant over-expression of lymphangiogenic receptor VEGFR-3 (in contrast to a non-statistically significant increase in hemangiogenic receptor VEGFR-2 expression). This study not only provides a link between ocular surface inflammation and the generation of T-cell mediated immunity in the lymphoid compartment, but also offers an example of how lymphangiogenesis and hemangiogenesis can be ‘naturally’ dissociated in a pathological state. Data suggests that these corneal lymphatics may serve as conduits for migration of corneal APCs to lymphoid tissues where they activate autoreactive T cells in DED.

Immunopathogenesis of DED: The pathogenesis is not fully understood. Ocular surface inflammation sustained by ongoing activation and infiltration of pathogenic immune cells. Strong evidence of T cell involvement. Recent work draining lymphoid tissue primary site for activation and generation of auto reactive effector T cells in DED (Chauhan et al; Role ofcTh17 cells in the immunopathogenesis of dry eye disease. Mucosal Immunol. 2009; 2 (4): 375-376.).

Expression levels of VEGF's and VEGFR's in DE corneas using RT PCR has demonstrated an increased transcript expression of VEGF-C, VEGF-D, and VEGFR-3. Thus, targeting pro-lymphangiogenic VEGF-C/D has therapeutic implications in DED.

Corneal lymphatics play an important role in mediating the corneal inflammation in dry eyes. Experiments: To validate this, inhibition of corneal neolymphangiogenesis was performed in a well characterized mouse model of DED described above. To see if inhibition of corneal neolymphangiogenesis could decrease ocular surface inflammation, anti-VEGF-C antibodies were administered i.p. daily from day-1 to day 10 to DED mice and assessed clinically using corneal fluorescein staining.

Methods (as described previously): Induction of Dry Eye Disease. Experimental Dry Eye Murine Model. Assessment of Corneal Surface: Corneal Fluorescein Staining. Immunohistochemistry: Monocyte/macrophage marker-CD11b; Pan-endothelial marker-CD31; Lymphatic endothelial marker-LYVE-1; Blood vessels: CD31^{hi}/LYVE-1; Lymph vessels: CD311/LYVE-1^{hi}. Morphometry of Lymphangiogenesis: Automated image analysis program written using Mat lab. Lymphatic Area (LA)-total surface area of the lymphatic vessels when projected into the plane of the image. Lymphatic Caliber (LC)-measure of the diameters of the lymphatic vessels.

Anti-VEGF-C antibody and treatment regimen. Experimental design: Three groups: Normal, DE group treated with IP normal Saline (Untreated) and DE group treated with anti-VEGF-C antibody (VGX-100; a gift from Vegenics, Australia). Daily IP application of anti-VEGF-C antibody/Normal saline from day-1 to day 13. Dose: 400 pg (20 mg/kg) in 100 μl of Normal Saline.

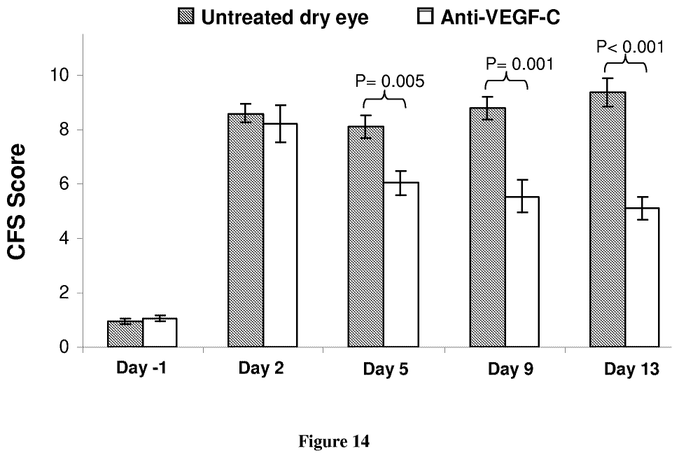

The results are presented in FIG. 14. Results: The data clearly shows a significant decrease in disease severity in anti-VEGF-C-treated group compared to the untreated group. In conclusion, suppression of lymphatic growth with VEGF-C blockade led to significant improvement in DED reflected by decrease in: corneal epitheliopathy; corneal infiltration of CD11b⁺ cells; expression of pro-lymphangiogenic growth factors and receptors (VEGF-C,-D, R3) in DE corneas; and mRNA expression levels of pro-inflammatory cytokines in the conjunctiva.

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

1. A method of treating dry eye disease (DED) in a human comprising:
administering a composition comprising at least one anti-lymphangiogenic agent and a pharmaceutically acceptable carrier to the eye of the human, in an amount effective to treat dry eye disease.

2. (canceled)

3. The method of claim 1, wherein the DED is an autoimmune DED or a DED associated with Sjogren's syndrome.

4. The method of claim 1, wherein the DED is DED due to excessively fast tear evaporation (evaporative dry eyes) or inadequate tear production.

5. The method of claim 1, wherein the dry eye disease is attributable to one or more causes selected from: aging, contact lens usage, medication usage.

6. The method of claim 1, wherein the dry eye disease is a complication of LASIK refractive surgery.

7. The method of claim 1, wherein the at least one anti-lymphangiogenic agent is purified or isolated.

8. The method of claim 1, wherein said at least one anti-lymphangiogenic agent is selected from the group consisting of: a nucleic acid molecule, an aptamer, an antisense molecule, an RNAi molecule, a protein, a peptide, a cyclic peptide, an antibody or antibody fragment, a polysaccharide, or a small molecule.

9-14. (canceled)

15. The method of claim 1, wherein the at least one anti-lymphangiogenic agent comprises a VEGF-C antibody or antigen binding fragment thereof, wherein the VEGF-C antibody or antigen-binding fragment thereof binds human VEGF-C and competitively inhibits the binding of antibody 69D09, produced by hybridoma ATCC PTA-4095, to human to VEGF-C.

16-18. (canceled)

19. The method of claim 1, wherein the at least one anti-lymphangiogenic agent is a human or humanized antibody.

20. (canceled)

21. The method of claim 1, wherein said composition further comprises a molecule that inhibits an activity of an inflammatory cytokine selected from the group consisting of IL-1, IL-7, IL23, IL-6 and TNF-α.

22. The method of claim 1, wherein the method further comprises administering an antibiotic to the human.

23. The method of claim 22, wherein the antibiotic is selected from the group consisting of amikacin, gentamicin, kanamycin, neomycin, netilmicin, streptomycin, tobramycin, teicoplanin, vancomycin, azithromycin, clarithromycin, dirithromycin, erythromycin, roxithromycin, troleandomycin, amoxicillin, ampicillin, azlocillin, carbenicillin, cloxacillin, dicloxacillin, flucloxacillin, meziocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, oflaxacin, trovafloxacin, mafenide, sulfacetamide, sulfamethizole, sulfasalazine, sulfisoxazole, trimethoprim, cotrimoxazole, demeclocycline, doxycycline, minocycline, oxytetracycline, tetracycline.

24. The method of claim 1, wherein the eye comprises at least one tissue or gland in or around the eye selected from the group consisting of ocular tissue, eyelids of the subject, ocular surface, meibomian gland and or lacrimal gland of the human.

25. The method of claim 1, wherein said composition is administered topically to the eye.

26. The method of claim 1, wherein said composition is in the form of a solid, a paste, an ointment, a gel, a liquid, an aerosol, a mist, a polymer, a film, an emulsion, or a suspension.

27. The method of claim 1, wherein the composition further comprises a compound selected from the group consisting of physiological acceptable salt, poloxamer analogs with carbopol, carbopol/hydroxypropyl methyl cellulose (HPMC), carbopol-methyl cellulose, carboxymethylcellulose (CMC), hyaluronic acid, cyclodextrin, and petroleum.

28. The method of claim 1, wherein said VEGF-C antibody or antigen binding fragment thereof binds to the same epitope as antibody 69D09.


SEQUENCE LISTING (54 sequences)

SEQ ID NO: 1 - protein, 9 aa, synthetic construct
  REGION 1..9: note = Synthetic peptide
SGYWWDTWF

SEQ ID NO: 2 - protein, 9 aa, synthetic construct
  REGION 1..9: note = Synthetic peptide
SCYWRDTWF

SEQ ID NO: 3 - protein, 9 aa, synthetic construct
  REGION 1..9: note = Synthetic peptide
KVGWSSPDW

SEQ ID NO: 4 - protein, 9 aa, synthetic construct
  REGION 1..9: note = Synthetic peptide
FVGWTKVLG

SEQ ID NO: 5 - protein, 9 aa, synthetic construct
  REGION 1..9: note = Synthetic peptide
YSSSMRWRH

SEQ ID NO: 6 - protein, 9 aa, synthetic construct
  REGION 1..9: note = Synthetic peptide
RWRGNAYPG

SEQ ID NO: 7 - protein, 9 aa, synthetic construct
  REGION 1..9: note = Synthetic peptide
SAVFRGRWL

SEQ ID NO: 8 - protein, 9 aa, synthetic construct
  REGION 1..9: note = Synthetic peptide
WFSASLRFR

SEQ ID NO: 9 - protein, 8 aa, synthetic construct
  REGION 1..8: note = Synthetic peptide
WQLGRNWI

SEQ ID NO: 10 - protein, 8 aa, synthetic construct
  REGION 1..8: note = Synthetic peptide
VEVQITQE

SEQ ID NO: 11 - protein, 8 aa, synthetic construct
  REGION 1..8: note = Synthetic peptide
AGKASSLW

SEQ ID NO: 12 - protein, 8 aa, synthetic construct
  REGION 1..8: note = Synthetic peptide
RALDSALA

SEQ ID NO: 13 - protein, 7 aa, synthetic construct
  REGION 1..7: note = Synthetic peptide
YGFEAAW

SEQ ID NO: 14 - protein, 7 aa, synthetic construct
  REGION 1..7: note = Synthetic peptide
YGFLWGM

SEQ ID NO: 15 - protein, 7 aa, synthetic construct
  REGION 1..7: note = Synthetic peptide
SRWRILG

SEQ ID NO: 16 - protein, 7 aa, synthetic construct
  REGION 1..7: note = Synthetic peptide
HKWQKRQ

SEQ ID NO: 17 - protein, 7 aa, synthetic construct
  REGION 1..7: note = Synthetic peptide
MDPWGGW

SEQ ID NO: 18 - protein, 7 aa, synthetic construct
  REGION 1..7: note = Synthetic peptide
RKVWDIR

SEQ ID NO: 19 - protein, 6 aa, synthetic construct
  REGION 1..6: note = Synthetic peptide
VWDHGV

SEQ ID NO: 20 - protein, 10 aa, synthetic construct
  REGION 1..10: note = Synthetic peptide
CWQLGRNWIC

SEQ ID NO: 21 - protein, 10 aa, synthetic construct
  REGION 1..10: note = Synthetic peptide
CVEVQITQEC

SEQ ID NO: 22 - protein, 10 aa, synthetic construct
  REGION 1..10: note = Synthetic peptide
CAGKASSLWC

SEQ ID NO: 23 - protein, 10 aa, synthetic construct
  REGION 1..10: note = Synthetic peptide
CRALDSALAC

SEQ ID NO: 24 - protein, 9 aa, synthetic construct
  REGION 1..9: note = Synthetic peptide
CYGFEAAWC

SEQ ID NO: 25 - protein, 9 aa, synthetic construct
  REGION 1..9: note = Synthetic peptide
CYGFLWGMC

SEQ ID NO: 26 - protein, 9 aa, synthetic construct
  REGION 1..9: note = Synthetic peptide
CSRWRILGC

SEQ ID NO: 27 - protein, 9 aa, synthetic construct
  REGION 1..9: note = Synthetic peptide
CHKWQKRQC

SEQ ID NO: 28 - protein, 9 aa, synthetic construct
  REGION 1..9: note = Synthetic peptide
CMDPWGGWC

SEQ ID NO: 29 - protein, 9 aa, synthetic construct
  REGION 1..9: note = Synthetic peptide
CRKVWDIRC

SEQ ID NO: 30 - protein, 8 aa, synthetic construct
  REGION 1..8: note = Synthetic peptide
CVWDHGVC

SEQ ID NO: 31 - protein, 13 aa, synthetic construct
  REGION 1..13: note = Synthetic peptide
CGQMCTVWCSSGC

SEQ ID NO: 32 - protein, 7 aa, synthetic construct
  VARIANT 4..6: note = X can be any naturally occuring amino acid
GYWXXXW

SEQ ID NO: 33 - protein, 8 aa, synthetic construct
  VARIANT 4..6: note = X can be any naturally occuring amino acid
  VARIANT 8: note = X can be any naturally occuring amino acid
GYWXXXWX

SEQ ID NO: 34 - protein, 448 aa, Homo sapiens
EVRLLESGGGLVQPGGSLRLSCAASGFTFRPRAMAWVRQAPGKGLEWVSSISAQGASAYYADSVKGRFTISRDNSKNTLYLQMNSLRAEDTAVYYCARDLSVSGFGPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 217 aa, Homo sapiens
SYELTQPPSSSGTPGQRVTISCSGSSSNIGRHTVSWYQQVPGTAPKLLIYSDDHRPSGVPDRFSASKSGTSASLTITGLQSEDEADYYCAAWDDSLNGPWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - DNA, 2292 bp, Homo sapiens
  source 1..2292: mol_type = genomic DNA
  CDS 1..2292
atggagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaa

SEQ ID NO: 37 - protein, 764 aa, Homo sapiens
MESKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 38 - DNA, 4195 bp, Homo sapiens
  source 1..4195: mol_type = genomic DNA
  CDS 20..3913
ccacgcgcagcggccggagatgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggacgctatctgtgcagcgtgtgcaacgccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcaccgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccctgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcggctgagagacggcacaaggatgagggccccggagctggccactcccgccatacgccgcatcatgctgaactgctggtccggagaccccaaggcgagacctgcattctcggagctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcaggtagctgaagcagagagagagaaggcagcatacgtcagcattttcttctctgcacttataagaaagatcaaagactttaagactttcgctatttcttctactgctatctactacaaacttcaaagaggaaccaggaggacaagaggagcatgaaagtggacaaggagtgtgaccactgaagcaccacagggaaggggttaggcctccggatgactgcgggcaggcctggataatatccagcctcccacaagaagctggtggagcagagtgttccctgactcct

SEQ ID NO: 39 - protein, 1298 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFR

SEQ ID NO: 40 - DNA, 4795 bp, Homo sapiens
  source 1..4795: mol_type = genomic DNA
  CDS 20..4111
ccacgcgcagcggccggagatgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggacgctatctgtgcagcgtgtgcaacgccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcaccgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccctgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcggctgagagacggcacaaggatgagggccccggagctggccactcccgccatacgccgcatcatgctgaactgctggtccggagaccccaaggcgagacctgcattctcggagctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcagctgtaaaggacctggccagaatgtggctgtgaccagggcacaccctgactcccaagggaggcggcggcggcctgagcggggggcccgaggaggccaggtgttttacaacagcgagtatggggagctgtcggagccaagcgaggaggaccactgctccccgtctgcccgcgtgactttcttcacagacaacagctactaagcagcatcggacaagacccccagcacttgggggttcaggcccggcagggcgggcagagggctggaggcccaggctgggaactcatctggttgaactctggtggcacaggagtgtcctcttccctctctgcagacttcccagctaggaagagcaggactccaggcccaaggctcccggaattccgtcaccacgactggccagggcacgctccagctgccccggcccctccccctgagattcagatgtcatttagttcagcatccgcaggtgctggtcccggggccagcacttccatgggaatgtctctttggcgacctcctttcatcacactgggtggtggcctggtccctgttttcccacgaggaatctgtgggtctgggagtcacacagtgttggaggttaaggcatacgagagcagaggtctcccaaacgccctttcctcctcaggcacacagctactctccccacgagggctggctggcctcacccacccctgcacagttgaagggaggggctgtgtttccatctcaaagaaggcatttgcagggtcctcttctgggcctgaccaaacagccaactagcccctggggtggccaccagtatgacagtattatacgctggcaacacagaggcagcccgcacacctgcgcctgggtgttgagagccatcctgcaagtctttttc

SEQ ID NO: 41 - protein, 1363 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 42 - protein, 10 aa, synthetic construct
  REGION 1..10: note = Synthetic peptide
  DISULFID 1..10: note = Cyclic peptide
CGYWLTIWGC

SEQ ID NO: 43 - protein, 13 aa, synthetic construct
  REGION 1..13: note = Synthetic peptide
  DISULFID 1..13: note = Disulfide linkage
CASELGKSTNTFC

SEQ ID NO: 44 - protein, 8 aa, synthetic construct
  REGION 1..8: note = Synthetic peptide
  DISULFID 1..8: note = Disulfide linkage
CNEESLIC

SEQ ID NO: 45 - protein, 11 aa, synthetic construct
  REGION 1..11: note = Synthetic peptide
  DISULFID 1..11: note = Disulfide linkage
CISVPLTSVPC

SEQ ID NO: 46 - protein, 132 aa, synthetic construct
  REGION 1..132: note = Synthetic peptide
MKLPVRLLVLMFWIPASSSDFVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPRTFGGGTKLEIKR

SEQ ID NO: 47 - protein, 137 aa, synthetic construct
  REGION 1..137: note = Synthetic peptide
MGWSGVFLFLLSGSTGVHSEIQLQQSGPDLVKPGASVKVSCRASGYSFTGYNMYWVKQSHGKSLEWIGYIDPYNGDTTYNQKFKGKATLTVDKSSSTAFMHLNSLTSEDSAVYYCARTSYYGGMDYWGQGTSVTVSS

SEQ ID NO: 48 - protein, 16 aa, synthetic construct
  REGION 1..16: note = Synthetic peptide
RSSQSLVHSNGNTYLH

SEQ ID NO: 49 - protein, 7 aa, synthetic construct
  REGION 1..7: note = Synthetic peptide
KVSNRFS

SEQ ID NO: 50 - protein, 9 aa, synthetic construct
  REGION 1..9: note = Synthetic peptide
SQSTHVPRT

SEQ ID NO: 51 - protein, 10 aa, synthetic construct
  REGION 1..10: note = Synthetic peptide
GYSFTGYNMY

SEQ ID NO: 52 - protein, 17 aa, synthetic construct
  REGION 1..17: note = Synthetic peptide
YIDPYNGDTTYNQKFKG

SEQ ID NO: 53 - protein, 9 aa, synthetic construct
  REGION 1..9: note = Synthetic peptide
TSYYGGMDY

SEQ ID NO: 54 - protein, 10 aa, synthetic construct
  REGION 1..10: note = Synthetic peptide
  MOD_RES 1..10: note = May be bound to form cyclic peptide
XGYWLTIWGX